SEQUENCE LISTING (29 sequences)

SEQ ID NO: 1 - RNA, 22 nt, Homo sapiens
  misc_RNA (1)..(22): miR1
uggaauguaaagaaguauguau

SEQ ID NO: 2 - RNA, 22 nt, Homo sapiens
  misc_RNA (1)..(22): miR146a
ugagaacugaauuccauggguu

SEQ ID NO: 3 - RNA, 23 nt, Homo sapiens
  misc_RNA (1)..(23): miR200b
uaauacugccugguaaugaugac

SEQ ID NO: 4 - RNA, 22 nt, Homo sapiens
  misc_RNA (1)..(22): miR320
aaaagcuggguugagagggcga

SEQ ID NO: 5 - RNA, 22 nt, Homo sapiens
  misc_RNA (1)..(22): miR144
ggauaucaucauauacuguaag

SEQ ID NO: 6 - RNA, 22 nt, Homo sapiens
  misc_RNA (1)..(22): miR450
uuuuugcgauguguuccuaaug

SEQ ID NO: 7 - RNA, 24 nt, Artificial Sequence
  Scrambled control
ucacaaccuccuagaaagaguaga

SEQ ID NO: 8 - DNA, 26 bp, Artificial Sequence
  Forward primer for Human VEGF 3′UTR mutation cloning
agagctccccggcgaagagaagagac

SEQ ID NO: 9 - DNA, 33 bp, Artificial Sequence
  Reverse primer for Human VEGF 3′UTR mutation cloning
tctagaaagcttggagggcagagctgagtgtta

SEQ ID NO: 10 - DNA, 26 bp, Artificial Sequence
  Forward primer for Rat VEGF 3′UTR mutation cloning
agagctcgggtcctggcaaagagaag

SEQ ID NO: 11 - DNA, 29 bp, Artificial Sequence
  Reverse primer for Rat VEGF 3′UTR mutation cloning
tcaagcttggagggcagagctgagtgtta

SEQ ID NO: 12 - DNA, 31 bp, Artificial Sequence
  Forward primer for Human fibronectin 3′UTR mutation cloning
agagctcatcatctttccaatccagaggaac

SEQ ID NO: 13 - DNA, 31 bp, Artificial Sequence
  Reverse primer for Human Fibronectin 3′UTR mutation cloning
tcaagctttaatcacccaccataattatacc

SEQ ID NO: 14 - DNA, 28 bp, Artificial Sequence
  Forward primer for Rat fibronectin 3′UTR mutation cloning
agagctctccagcccaagccaacaagtg

SEQ ID NO: 15 - DNA, 30 bp, Artificial Sequence
  Reverse primer for Rat fibronectin 3′UTR mutation cloning
tcaagctttccacagtagtaaagtgttggc

SEQ ID NO: 16 - DNA, 20 bp, Artificial Sequence
  Forward primer ANP
ctgctagaccacctggagga

SEQ ID NO: 17 - DNA, 20 bp, Artificial Sequence
  Reverse primer ANP
aagctgttgcagcctagtcc

SEQ ID NO: 18 - DNA, 20 bp, Artificial Sequence
  Forward primer BNP
gacgggctgaggttgtttta

SEQ ID NO: 19 - DNA, 20 bp, Artificial Sequence
  Reverse primer BNP
actgtggcaagtttgtgctg

SEQ ID NO: 20 - DNA, 20 bp, Artificial Sequence
  Forward primer 18S rRNA
gtaacccgttgaaccccatt

SEQ ID NO: 21 - DNA, 19 bp, Artificial Sequence
  Reverse primer (18S rRNA)
ccatccaacggtagtagcg

SEQ ID NO: 22 - DNA, 20 bp, Artificial Sequence
  Forward primer (_-actin)
catcgtactcctgcttgctg

SEQ ID NO: 23 - DNA, 20 bp, Artificial Sequence
  Reverse primer (_-actin)
cctctatgccaacacagtgc

SEQ ID NO: 24 - DNA, 21 bp, Artificial Sequence
  Forward primer (ET-1)
aagccctccagagagcgttat

SEQ ID NO: 25 - DNA, 21 bp, Artificial Sequence
  Reverse primer (ET-1)
ccgaaggtctgtcaccaatgt

SEQ ID NO: 26 - DNA, 26 bp, Artificial Sequence
  Forward primer (VEGF)
ggcctccgaaaccatgaactttctgc

SEQ ID NO: 27 - DNA, 27 bp, Artificial Sequence
  Reverse primer (VEGF)
tgcatgccctcctgcccggctcaccgc

SEQ ID NO: 28 - DNA, 20 bp, Artificial Sequence
  Forward primer (fibronectin)
gataaatcaacagtgggagc

SEQ ID NO: 29 - DNA, 20 bp, Artificial Sequence
  Reverse primer (fibronectin)
cccagatcatggagtcttta

(19) United States
(12) Patent Application Publication
(10) Pub. No.: US 2012/0282326 A1
(43) Pub. Date: Nov. 8, 2012

(54) COMPOSITIONS AND METHODS RELATED TO MIRNA IN DIABETIC CONDITIONS

(75) Inventors: Subrata Chakrabarti, London (CA); Biao Feng, London (CA); Shali Chen, London (CA); Yuexiu Wu, London (CA); Kara Rozsa McArthur, London (CA)

(73) Assignee: THE UNIVERSITY OF WESTERN ONTARIO, London, ON (CA)

(21) Appl. No.: 13/514,829

(22) PCT Filed: Dec. 16, 2010
(86) PCT No.: PCT/CA10/02005

§ 371 Date: Jul. 3, 2012

Related U.S. Application Data
(60) Provisional application No. 61/286,875, filed on Dec. 16, 2009.

(51) Int. Cl. A61K 31/7105; A61K 31/711; A61K 9/127; A61K 9/14; A61P 27/02; C12Q 1/68; G01N 33/53; A61K 31/713; A61P 3/10; A61P 9/10; A61K 31/7088; C40B 30/04

(52) U.S. Cl. 424/450; 514/44 A; 514/44 R; 424/400; 506/9; 435/6.12; 435/6.11; 424/93.2

(57) ABSTRACT

The present invention relates to methods of treating a disorder associated with glucose mediated cell damage in a subject comprising administering to the subject an agent that modulates the expression of one or more miRNAs in a damaged cell or cells of the subject. The present invention also relates to compositions for treating a disorder associated with glucose mediated cell damage comprising an agent that modulates the expression of one or more miRNAs in a damaged cell or cells. The invention also relates to methods of diagnosing a disorder associated with glucose mediated cell damage in a subject, including diagnosis of diabetic retinopathy.

### FIELD OF THE INVENTION

The present invention relates to microRNA molecules and microRNA profiles of conditions or disorders associated with glucose-mediated cell damage, including chronic diabetes.

### BACKGROUND OF THE INVENTION

Throughout this document, various references are cited in square brackets to describe more fully the state of the art to which this invention pertains.

### Diabetic Retinopathy

Nearly all patients with type I diabetes and 60% of patients with type II diabetes develop retinopathy. By 2030, an estimated 366 million people worldwide will become affected by diabetes. In Canada, over 2 million people suffer from this disease, of which ˜10% is type 1 and ˜90% is type 2 diabetic. Diabetic retinopathy (DR) is the most important systemic cause of blindness in North America. Alteration of several protein molecules have been demonstrated in DR. The targeting of individual proteins for the treatment of diabetic retinopathy has been tried for a long time, but so far all efforts have failed in clinical trials. Endothelial damage is a key feature in all chronic diabetic complications, including DR. Uptake of glucose by endothelial cells, which line the walls of the blood vessels, is not dependent on insulin. In diabetes, when blood glucose levels become high, glucose flows into the endothelial cells causing injury to these cells.

There are two types, or stages of retinopathy: non-proliferative and proliferative. During the non-proliferative diabetic retinopathy, blood vessels in the eye become larger in certain spots (microaneurysms). Blood vessels may also become blocked. There may be small amounts of bleeding (retinal hemorrhages), and fluid may leak into the retina. The proliferative stage is the more advanced and severe form of the disease. New blood vessels start to grow in the eye (angiogenesis). These new vessels are fragile and can bleed (hemorrhage). Small scars develop, both on the retina and in other parts of the eye (the vitreous). The end result is vision loss, as well as other problems.

### MicroRNA

MicroRNAs (“miRNA”) are recently identified naturally occurring molecules. miRNAs are small (˜20-25 nucleotide) RNA molecules that have significant effects on the regulation of gene expression [1]. Transcription of miRNA occurs through RNA polymerase II, creating primary miRNAs with 5′ caps and poly-A tails. The processing of primary miRNAs to precursor miRNAs (70-100 nucleotides, and hairpin-shaped) in the nucleus is mediated by RNAse II, Drosha and DGCR8. Following their synthesis, precursor miRNAs are exported to the cytoplasm by Exportin 5. In the cytoplasm, precursor miRNAs are further processed by RNAse III Dicer into mature miRNA, the functionally active form [1, 2]. miRNA along with RISC complex, binds to specific mRNA targets and causes degradation of specific mRNA or translational repression [1, 2]. Several investigators using overexpression experiments have demonstrated the importance of miRNA in diverse cellular processes. miRNAs are also thought to play important roles in controlling histone modification [3]. A significant number of miRNA coding regions are located in the intron of the protein coding gene and are believed to be co-regulated with their host genes. However, it is also possible that they are regulated by their own promoters. Several miRNAs have been identified in malignancies. These have been demonstrated to regulate a wide variety of factors, including oncogenes (c-MYC), transcription factors (NFκB) and methylation [1, 2].

There is considerable interest in the scientific community as to the potential therapeutic applications of miRNA in various diseases. From a mechanistic standpoint, one miRNA regulates multiple genes, hence targeting one or few miRNAs could potentially provide a unique opportunity to prevent multiple gene expression. Such RNA-based therapy is attractive due to the specificity of action of the target miRNAs.

Deregulation of miRNA expression may be a cause of disease, and detection of expression of miRNA may become useful as a diagnostic.

miRNA in Diabetes

There are no studies in the literature that have characterized alterations of specific miRNAs in the retina of diabetic individuals. However, studies in other diabetic complications have demonstrated miRNA alterations. For example, alteration of miR375, involved in glucose induced insulin gene expression, has been demonstrated in diabetes [4]. Up-regulation of miR320 has been demonstrated in cardiac microvascular endothelial cells in type 2 diabetic rats [5]. miR377 has been shown to regulate increased fibronectin production in diabetic nephropathy by modulating p21-activated kinase and superoxide dismutase [6]. mi192 has also been shown to be altered in diabetic nephropathy by influencing TGFβ induced collagen expression [7]. Hearts from diabetic rabbit demonstrated reduced miR133, which modulates HERO K+ channel causing QT prolongation [8]. Furthermore, miR1 has been implicated in glucose induced cardiomyocyte apoptosis. Recent studies by the Applicants have demonstrated that hearts from diabetic rats and cardiomyocytes exposed to glucose show down-regulation of miR133a, which is directly linked to cardiomyocyte hypertrophy [9]. Other studies in non-diabetic cardiac hypertrophy have demonstrated down regulation of miR1 and miR133 [10,11].

WO 2009/045356 (WO '356) discloses methods for treating disorders by administering miRNAs to alter the ability of vascular endothelial growth factor (VEGF) to induce cellular and tissue responses or changes. WO '356 discloses methods for treating wound healing and disorders like cancer, inflammation and macular degeneration. However, WO '356 does not disclose which miRNAs are altered in cells exposed to glucose, or in the retina of diabetic subjects.

There is a need in the art for an efficient therapeutic method for the treatment of conditions or disorders associated with glucose mediated cell damage including chronic diabetic disorders such as diabetic retinopathy. There is also a need in the art for a robust, early stage detection of diabetic conditions.

### SUMMARY OF THE INVENTION

In one aspect, the present invention provides a method of treating a subject of a disorder associated with glucose mediated cell damage characterized in that said method comprises administering to the subject an agent or a mixture of agents that modulates the expression of one or more miRNAs in a cell or cells of the subject in need thereof.

In another aspect the present invention provides for a composition for treating a disorder associated with glucose mediated cell damage comprising an agent or mixture of agents that modulates the expression of one or more miRNAs in a cell or cells in need thereof, and a pharmaceutically acceptable carrier.

In aspects of the invention the agent or mixture of agents up-regulates the expression of at least one miRNA of the one or more miRNAs in the cell or cells of the subject in need thereof. In one aspect, the at least one miRNA of the one or more miRNAs is selected from miR-1, miR-146a, miR200b or miR-320.

In aspects of the invention the agent or mixture of agents comprises an oligonucleotide or mixture of oligonucleotides.

In aspects of the invention the oligonucleotide or mixture of oligonucleotides is provided as a miRNA, a derivative or analog thereof, a miRNA precursor, a mature miRNA or a DNA molecule encoding for said at least one miRNA.

In aspects of the invention the oligonucleotide or mixture of oligonucleotides is provided in a composition comprising a pharmaceutically acceptable carrier.

In aspects of the invention the oligonucleotide or mixture of oligonucleotides is selected from SEQ ID NOs. 1-4.

In aspects of the invention the agent or mixture of agents is provided within a delivery vehicle.

In aspects of the invention the delivery vehicle is selected from a viral vector, microspheres, liposomes, colloidal gold particles, lipopolysaccharides, polypeptides, polysaccharides, or pegylation of viral vehicles.

In aspects of the invention the agent or mixture of agents down-regulates the expression of at least one miRNA of the one or more miRNAs in the cell or cells in need thereof. In one aspect the at least one miRNA of the one or more miRNAs is selected from miR-144, or miR-450.

In aspects of the invention the agent or mixture of agents comprises an inhibitor or mixture of inhibitors of the at least one miRNA.

In aspects of the invention the inhibitor or mixture of inhibitors is selected from an antagomir, an antisense RNA or a short interfering RNA.

In aspects of the invention the inhibitor or mixture of inhibitors is provided in a composition comprising a pharmaceutically acceptable carrier.

In aspects of the invention the disorder is a chronic diabetic condition, including diabetic retinopathy, diabetic nephropathy, or diabetic large vessels disease.

In aspects of the invention the agent or mixture of agents is administered by a parenteral administration route or a topical route.

In aspects of the invention said disorder is diabetic retinopathy, and the agent or mixture of agents is administered by intraocular administration or topical instillation to the eye.

In aspects of the invention said disorder is diabetic retinopathy, and the agent or mixture of agents is administered by an ocular implant.

In another aspect the present invention provides for a method of treating diabetic retinopathy in a subject, characterized in that said method comprises administering to the subject a composition comprising at least one of: (a) an oligonucleotide targeted to miR-1, miR-146a, miR-200b or miR-320, and (b) an inhibitor of miR-144 or miR-450.

In another aspect the present invention provides for a method for diagnosing a disorder in a subject, said disorder associated with glucose mediated cell damage, characterized in that said method comprises measuring an expression profile of one or more miRNAs in a sample from the subject, wherein a difference in the miRNA expression profile of the sample from the subject and the miRNA expression profile of a normal sample or a reference sample is indicative of the disorder associated with glucose mediated cell damage.

In one aspect of the method for diagnosing a condition of the present invention, the disorder is a chronic diabetic complication, including diabetic retinopathy, diabetic nephropathy, or diabetic large vessels disease.

In one aspect of the method for diagnosing a condition of the present invention, the method is a method of diagnosing diabetic retinopathy in the subject.

In one aspect of the method for diagnosing a condition of the present invention the one or more miRNAs are selected from miR1, miR146a, miR200b, miR320, miR144 or miR450.

In one aspect of the method for diagnosing a condition of the present invention, down-regulation of miR1, miR146a, miR200b and miR320, and up-regulation of miR144 and miR450 with respect to the normal sample or reference sample is indicative of the disorder.

The present invention has the following advantages:

(a) miRNAs are natural agents and when used as drugs are very specific,
(b) miRNAs can be easily synthesized,
(c) miRNA can be delivered by intraocular injections in the vitreous. Intravitreal drug delivery is an accepted way to treat retinal diseases, and
(d) miRNAs can be used in the diagnosis of diabetes and diabetic complications, including diabetic retinopathy. This provides a new method of diagnosing these conditions at a very early stage.

### BRIEF DESCRIPTION OF THE DRAWINGS

The invention will be better understood and objects of the invention will become apparent when consideration is given to the following detailed description thereof. Such description makes reference to the annexed drawings wherein:

FIG. 1 Graph showing a miRNA array-volcano plot, showing miRNA alteration in control vs. streptozotocin (STZ) induced (a model of type I diabetes) diabetic (treated) rat retina.

FIG. 2 a): Graph showing quantitative real time polymerase chain reaction (qRT-PCR) analysis of the expression levels of miR320 in endothelial cells exposed to low glucose (LG) and high glucose (HG). b)-d): Graphs showing qRT-PCR analysis of the expression levels of (b) fibronectin (FN) mRNA, (c) endothelin-1 (ET-1) mRNA and (d) vascular endothelial growth factor (VEGF) mRNA in endothelial cells exposed to: high glucose (HG), low glucose (LG), and in endothelial cells exposed to HG and transfected with a negative miRNA (HG+neg) or with a miR320 mimic (HG+miR320). e): Graph showing MAPK (ERK1/2) activation in endothelial cells exposed to LG, HG, LG or HG and negative transfection (LG+Neg, HG+Neg), and LG or HG and miR320 mimic transfection (LG+miR320, HG+miR320). *=statistically significant difference compared to LG.

FIG. 3. a): Graph showing qRT-PCR analysis of the expression level of miRNA 146a in human umbilical vein endothelial cells (HUVECs) exposed to 25 mmol/L glucose compared to 5 mmol./L glucose and in endothelial cells exposed to 25 mmol/L glucose and transfected with a negative miRNA (25 mM+Scram) or with a miR146a mimic (25 mM+miR146a). b)-c): Graphs showing qRT-PCR and ELISA analysis of the expression levels of b) fibronectin (FN) mRNA and c) fibronectin protein levels in HUVECs exposed to 5 mmol/L glucose, 25 mmol/L glucose, and to HUVECs exposed to 25 mmol/L glucose and transfected with a negative (scrambled) miRNA (25 mM+Scram) or with a miR146a mimic (25 mM+miR146a). Scram=scrambled, *=statistically significant difference from 5 mM or 5 mM scram; **=significantly different from 25 mM or 25 mM scram; miRNA levels are expressed as a ratio of RNU6B (U6) and normalized to LG; mRNA expressed as a ratio to 18S RNA and normalized to LG.

FIG. 4. a): Graph showing miR146a levels in retinal tissues from the STZ induced diabetic rats (D) compared to miR146a levels in age and sex matched controls (C). b): graph showing the efficiency of intravitreal delivery in which intravitreal injection of miR146a mimic (D+146a) leads to increased retinal miR146a but not scrambled mimic (D+SC). c)-d): Graphs showing fibronectin (FN) (c) mRNA and (d) protein levels in retinal tissues from the STZ diabetic rats. Diabetes induced FN mRNA and protein up-regulation were prevented by intravitreal injection of miR146a mimic (D+miR146a) but not by scrambled controls (D+Sc). Data expressed as a ratio of I, c) RNU6(U6), d) 185, *=statistically significant difference than corresponding C, n=5/group.

FIG. 5. a): Alignment of fibronectin (FN1) 3′UTR sequence with mature miR146a based on bioinformatics predictions (www.TargetScan.org, www.microrna.org, www.ebi.ac.uk1). b)-c): Graphs showing binding of miR146a with (b) human and (c) rat FN1 promoter Luciferase reporter assay. **=statistically significant difference from vector alone or Vector+scrambled).

FIG. 6. a): microphotograph of LNA™-ISH study of retinal tissues in a control rat retina showing localization of miR146a. (b): Higher magnification micrograph of panel (a) showing positive staining in the retinal capillaries (arrow). c): microphotograph of LNA™-ISH study of retinal tissues in a STZ diabetic rat showing minimum (if any) expression of miR146a in the capillaries (arrow). Alkaline phosphatase (ALK Phos) was used as chromogen with no counterstain.

FIG. 7. Graph showing NF-kB activity in HUVECs. The X axis shows various conditions: HUVECs exposed to 5 mmol/L glucose, 25 mmol/L glucose, and to HUVECs exposed to 25 mmol/L glucose and transfected with a negative miRNA (25 mM+Scram) or with a miR146a mimic (25 mM+miR146a). *=Statistically significant difference from other groups. The data were normalized to 5 mM glucose group.

FIG. 8.: Graph showing miR146a levels in retinal tissues from the db/db diabetic mice (db/db) (a model of type 2 diabetes) compared to miR146a levels in age and sex matched controls (C). Data expressed as a ratio of RNU6 (U6), *=statistically significant difference from the other group.

FIG. 9. Graphs showing microRNA and VEGF alteration in the rat retina in diabetes. a) qRT-PCR and b) ELISA analysis from non-diabetic control and diabetic (STZ induced, after 1 month of follow-up) rat retinal tissue samples showing increased levels of VEGF mRNA and protein in the retina of the diabetic rat. c): Graph showing qRT-PCR of miR200b in the retina of diabetic rats compared to the non-diabetic controls. miRNA data are expressed a ratio to RNU6B (U6) and normalized to controls; (mRNA levels are expressed as a ratio to 18S RNA, and normalized to controls. *=Statistically significant difference from the other group).

FIG. 10: Effects of glucose induced miR200b down-regulation in HUVECs. a) expression of miRNA 200b in HUVECs when exposed to 25 mmol/L. glucose (HG), 5 mmol./L glucose (LG), and 25 mM L-glucose (osmotic control, OSM). b) VEGF mRNA expression in HUVEC when exposed to LG, HG, 25 mM L-glucose (OSM), and when transfected with scrambled (scr) mimics or miR200b and exposed to LG (LG+Scr; LG+200b) or to HG (HG+Scr; HG+200b) and transfected with antagomirs [200b(A)] and exposed to LG [LG+200b (A)]. c) Efficiency of miR200b mimic transfection as shown by increased miR200b expression in HUVECs exposed to HG following miR200b mimic transfection compared to scrambled (scr) mimics. d) Similar to HUVECs, bovine retinal endothelial cells (BREC) showed glucose induced miR200b down-regulation e) Transfection of BRECs with miR200b mimics [using miR200b cloned in PcDNA3.1 vector (V200b), but not by empty vector (V)] normalized HG induced up-regulation of VEGF mRNA expression. f) Efficiency of miR-200b mimic transfection in the BREC was shown by increased miR200b expression in these cells following miR-200b mimic transfection compared to vector control. (LG=5 mM glucose, Scr=scrambled miRNA, 200b=miR200b mimic, 200b(A)=200b antagomir, OSM=25 mML glucose [osmotic control]. *=significantly different from LG or LG scram, +=significantly different from HG or HG scram. miRNA levels are expressed as a ratio of RNU6B (U6) and normalized to LG; mRNA expressed as a ratio to 18S RNA and normalized to LG.

FIG. 11: HG induced and VEGF-mediated increased transendothelial permeability a), duration dependent data and b) at the endpoint) were prevented by miR200b mimic (200b) transfection but not by scrambled (scr) mimic. c) Similarly, glucose induced EC tube formation was prevented by miR200b mimic transfection but not by scrambled (scr) mimic. d) shows the quantification of the tube formation assay.

(LG=5 mM glucose, Scr=scrambled miRNA, 200b=miR200b mimic, 200b(A)=200b antagomir, OSM=25 mML glucose (osmotic control). *=significantly different from LG or LG scram, +=significantly different from HG or HG scram. miRNA levels are expressed as a ratio of RNU6B (U6) and normalized to LG; mRNA expressed as a ratio to 18S RNA and normalized to LG.

FIG. 12. a) Alignment of VEGF 3′UTR (and mutated [mut] VEGF3′-UTR) sequence with mature miR200b based on bioinformatics predictions (www.TargetScan.org, www.microrna.org, www.ebi.ac.uk1). b) (human), e) (rat): Graphs showing binding of miR200b with VEGF promoter Luciferase reporter assay showing dose dependent binding of VEGF 3′UTR with miR200b. Relative promoter activities were expressed as luminescence units normalized for β-galactosidase expression, *=statistically significant difference from vector alone or Vector+scrambled).

FIG. 13. Graphs showing miR200b mediated alteration of retinal VEGF and its prevention by miR200b. a) VEGF mRNA and b) protein levels in the control (Cont) and diabetic (Diab) rat (STZ induced) retina with or without intravitreal injection of miR200b mimic (200b) and scrambled mimic (Scr). c): Efficiency of intravitreal delivery as demonstrated by increased retinal miR200b expression following intravitreal injection of miR200b mimic compared to scrambled mimic. *=statistically significant difference from control or Diabetic+scr (in right graph), +=significantly different from diabetic.

FIG. 14. Photomicrographs showing functional consequences of miR200b mediated alteration of retinal VEGF in diabetes. a): photomicrograph showing LNA™-ISH study of retinal tissues in a control rat retina showing localization of miR200b in the endothelium of retinal capillaries (arrow), ganglion cells (arrowheads) and in the cells of inner nuclear layer (double arrowheads, both in the glial and neuronal elements; inset shows enlarged view of capillaries with cytoplasmic and nuclear miR200b localization (arrow). b): photomicrograph showing LNA™-ISH study of retinal tissues in a diabetic rat (STZ induced) retina (in similar orientation as in panel (a)) showing minimum (if any) expression of miR200b). c): photomicrograph showing immunocytochemical stain on the control rat retina using anti-albumin antibody showing intra vascular albumin (arrow). d): photomicrograph showing similar immunocytochemical stain as in panel (c) in the diabetic rat (STZ induced) retina resulted in intravascular reactivity (arrow) and diffuse staining of the retina, indicating increased vascular permeability. e): photomicrograph showing albumin staining was only present in the intravascular compartment (arrow) following intravitreal miR200b injection in the retina of the STZ induced diabetic rats. ALK Phos was used as chromogen with no counterstain in LNA™-ISH; DAB chromogen and hematoxylin counterstain in albumin stain.

FIG. 15. Graphs showing mir200b regulation of diabetes induced p300 alteration. a): graph showing p300 mRNA up-regulation in the HUVECs under different conditions: HUVECs exposed to 5 mmol/L glucose (LG), 25 mmol/L glucose (HG), and to HUVECs exposed to 25 mmol/L glucose and transfected with a negative miRNA (HG+Scram) or with a miR200b mimic (HG+200b) b): graph showing miR200b expression in HUVECs. No effects of p300 siRNA transfection on miR200b expression were seen. c): graph showing retinal P300 mRNA expression in the diabetic rats (compared to the controls). *=statistically significant difference from control or LG+=significantly different from diabetic or HG, scram=scrambled control.

FIG. 16. a): photomicrograph showing LNA™-ISH study of retinal tissues from non-diabetic human retina showing localization of miR200b in the retinal capillaries (arrow), and in the cells of inner nuclear layer (double arrowheads). Inset shows high power pictures of microvessels with endothelial stain (arrow). b): photomicrograph of a diabetic human retina (in similar orientation as in panel a)) showing minimal (if any) expression of miR200b. c): photomicrograph showing immunocytochemical stain on the non-diabetic human retina using anti-albumin antibody showing presence of intravascular albumin (arrow). d): photomicrograph of diabetic human retina showing intravascular albumin staining (arrow) and diffuse staining in the retina, indicating increased vascular permeability. ALK Phos was used as chromogen with no counterstain in LNA™-ISH; DAB chromogen and hematoxylin counterstain in albumin stains.

FIG. 17 illustrates qRT-PCR analysis of the expression levels of miR200b in mice retina of control and diabetic (db/db) mice—a model of type 2 diabetes. *=Statistically significant difference from control.

FIG. 18 Graph showing qRT-PCR analysis of the expression levels of miR1 in rat retina of control and diabetic animals. A statistically significant decrease in the expression of miR1 in the retina of diabetic rats compared to the retinas of normal (control) rats. *=p<0.05 compared to control.

FIG. 19 a): graph showing qRT-PCR analysis of the expression levels of miR144 in control and diabetic rat (streptozotocin induced, model of type 1 diabetes) retinal tissue samples. b): graph showing qRT-PCR analysis of the expression levels of miR450 in control and diabetic rat retinal tissue samples. *=p<0.05 compared to control.

FIG. 20 graph showing amplification plots (qRT-PCR analysis) of vitreous fibrovascular tissue from two patients with proliferative diabetic retinopathy showing presence of miR146a and miR320.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION OF THE INVENTION

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context, clearly indicates otherwise (for example “including”, “having” and “comprising” typically indicate “including without limitation”). Singular forms including in the claims such as “a”, “an” and “the” include the plural reference unless expressly stated otherwise.

The invention will be explained in detail by referring to the figures.

The present invention relates to the discovery of altered levels of microRNAs (miRNA) in cells exposed to relatively high glucose levels and in the retina of diabetic subjects.

The Applicants discovered that certain miRNAs' profiles may be altered in cells exposed to relatively high levels of glucose and in the cells of diabetic subjects, including in the retina of diabetic subjects. The Applicants further discovered that the altered miRNAs may correlate with the up- and down-regulation of proteins known to play important roles in diabetes. Furthermore, the Applicants discovered that miRNA molecules may be used to substantially normalize the levels of the mRNAs that translate for said proteins.

The present invention relates to methods and compositions of treating a disorder associated with glucose mediated cell damage in a subject. The methods may comprise administering to the subject an agent or mixture of agents that modulate the expression of one or more miRNAs in a cell or cells of the subject in need thereof. The compositions may comprise an agent or mixture of agents that modulate the expression of one or more miRNAs in a cell or cells in need thereof.

As such, the present invention relates to miRNA-based compositions and miRNA-based methods which may be useful for substantially normalizing the expression of mRNAs and the production of proteins that may be either down-regulated or up-regulated in cells exposed to relatively high glucose levels and in cells of subjects having a disorder related to exposure to blood glucose levels, such as diabetes, including in the retina of subjects with diabetes. The present invention also relates to diagnostic methods based on the different expression profiles of miRNAs in diabetes-related disorders.

### MicroRNA

miRNAs are complementary to a part or fragment of one or more mRNAs. Moreover, miRNAs do not require absolute sequence complementarity to bind a mRNA, enabling them to regulate a wide range of target transcripts. miRNAs typically bind to target sequences with gaps between matched nucleotides. As used herein, the term “absolute sequence complementarity” is meant to describe a requirement that each nucleotide pair along the length of two sequences, e.g. a miRNA and a target gene or transcript, bind without gaps. The term “complementary” is meant to describe two sequences in which at least about 50% of the nucleotides bind from one sequence to the other sequence in trans.

miRNAs are frequently complementary to the 3′ UTR of the mRNA transcript, however, miRNAs of the invention may bind any region of a target mRNA. Alternatively, or in addition, miRNAs target methylation genomic sites which correspond to genes encoding targeted mRNAs. The methylation state of genomic DNA in part determines the accessibility of that DNA to transcription factors. As such, DNA methylation and de-methylation regulate gene silencing and expression, respectively.

miRNAs of the invention include the sequences in Table 1 (SEQ ID NOs. 1-6) and to homologs and analogs thereof, to miRNA precursor molecules, and to DNA molecules encoding said miRNAs.

TABLE 1

| | |
|---|---|
| miR1 | SEQ ID NO. 1 |
| miR146a | SEQ ID NO. 2 |
| miR200b | SEQ ID NO. 3 |
| miR320 | SEQ ID NO. 4 |
| miR144 | SEQ ID NO. 5 |
| miR450 | SEQ ID NO. 6 |

Preferably the identity of a homolog to a sequence of SEQ ID NOs 1-6 may be at least 90%, more preferably at least 95% identical.

Further, the invention encompasses nucleotide sequences, which may hybridize under stringent conditions with the nucleotide sequence of SEQ ID NOs 1-6, a complementary sequence thereof or a highly identical sequence thereof. Stringent hybridization conditions may comprise washing for 1 h in 1×SSC and 0.1% SDS at 45° C., preferably at about 48° C. and more preferably at about 50° C., particularly for about 1 h in 0.2×SSC and 0.1% SDS.

It should be noted that mature miRNAs may usually have a length of about 19-24 nucleotides (and any range in between), particularly 21, 22 or 23 nucleotides. The miRNAs, however, may be also provided as a precursor which may have a length of about 70 to about 100 nucleotides (pre-miRNA). It should be noted that the precursor may be produced by processing of a primary transcript which may have a length of greater than about 100 nucleotides (pri-miRNA).

The miRNA as such may usually be a single-stranded molecule, while the miRNA-precursor may usually be in the form of an at least partially self-complementary molecule capable of forming double-stranded portions, e.g. stem- and loop-structures. DNA molecules encoding the miRNA, pre-miRNA and pri-miRNA molecules may also be encompassed by the invention. The nucleic acids may be selected from RNA, DNA or nucleic acid analog molecules, such as sugar- or backbone-modified ribonucleotides or deoxyribonucleotides. It should be noted, however, that other nucleic analogs, such as peptide nucleic acids (PNA) or locked nucleic acids (LNA), may also be suitable.

The nucleic acid molecules of the invention may be obtained by chemical synthesis methods or by recombinant methods, e.g. by enzymatic transcription from synthetic DNA-templates or from DNA-plasmids isolated from recombinant organisms. Typically phage RNA-polymerases are used for transcription, such as T7, T3 or SP6 RNA-polymerases.

The invention may also relate to a recombinant expression vector comprising a recombinant nucleic acid operatively linked to an expression control sequence, wherein expression, i.e. transcription and optionally further processing results in a miRNA-molecule or miRNA precursor (pri- or pre-miRNA) molecule as described above. The vector may be an expression vector suitable for nucleic acid expression in eukaryotic, more particularly mammalian cells. The recombinant nucleic acid contained in said vector may be a sequence which results in the transcription of the miRNA-molecule as such, a precursor or a primary transcript thereof, which may be further processed to give the miRNA-molecule.

miRNA Modulators

miRNAs may act as targets for therapeutic procedures, e.g. inhibition or activation of miRNA may modulate a process like angiogenesis. Compositions and methods of the invention may include one or a mixture of agents such as a miRNA molecule, a molecule that augment the levels of a miRNA, and/or an inhibitors of a miRNAs that modifies or decreases the production of a peptide or the ability of the peptide to induce a response in at least one cell of a subject. As used in this document, the term “miRNA modulators” includes molecules or compounds that augment, reduce or attenuate the levels of a miRNA, and/or an inhibitor of a miRNA.

Contemplated agents which may act as miRNA modulators may include miRNA molecules, single or double-stranded RNA or DNA polynucleotides, peptide nucleic acids (PNAs), proteins, small molecules, ions, polymers, compounds, antibodies, intrabodies, antagomirs or any combination thereof. miRNA modulators may augment, reduce, attenuate or inhibit miRNA expression levels, activity, and/or function. One exemplary miRNA inhibitor may be an antagomir. Antagomirs of the invention may be chemically engineered oligonucleotides that specifically and effectively silence the expression of one or more miRNA(s). Antagomirs may be cholesterol-conjugated single-stranded RNA molecules of about 21-23 nucleotides in length and are complementary to at least one mature target miRNA.

miRNA inhibitors of the invention may repress or silence the expression or function of an endogenous or exogenous miRNA gene by, for example, targeting a genomic sequence, precursor sequence, and preventing transcription of the gene, or the miRNA itself, or causing degradation of the miRNA or its precursor. For example, an inhibitor may be an interfering RNA (RNAi), short interfering RNA (siRNA), short hairpin RNA (shRNA), microRNA (miRNA), double-stranded RNA (dsRNA), antisense oligonucleotide (RNA or DNA), morpholino, or peptide nucleic acid (PNA). In one aspect, the miRNA inhibitor may be a single-stranded RNA, DNA or PNA that binds to the miRNA, creating a dsRNA, DNA/RNA hybrid, or RNA/PNA hybrid that may be subsequently degraded. In an alternate or additional aspect, the inhibitor may be a single-stranded RNA, DNA or PNA that binds to the miRNA, which creates a dsRNA, DNA/RNA hybrid, or RNA/PNA hybrid and prevents the miRNA from binding to a target sequence.

A miRNA inhibitor may be between about 17 to 25 nucleotides in length (and any range in between) and comprises a 5′ to 3′ sequence that is at least 90% complementary to the 5′ to 3′ sequence of a mature miRNA.

In another aspect of the invention, miRNA inhibitors may be tagged with sequences or moieties that cause the miRNA to be degraded or sequestered into a cellular compartment or organelle such that the miRNA may not bind a target sequence. For instance, the miRNA inhibitor may be tagged with a secretory signal that causes the miRNA to be expelled from the cell. Alternatively, or in addition, the miRNA inhibitor may be tagged with a ubiquitin tag that causes the miRNA to be degraded.

miRNA inhibitors may reduce the ability of a miRNA to decrease the translation of a polypeptide in a cell or tissue, for example, in an additive capacity. In another aspect of the invention, a miRNA inhibitor may reduce the ability of a miRNA to decrease the translation of a polypeptide in a cell or tissue, for example, in a synergistic capacity.

In aspects, the agent that may act as a miRNA modulator may be an RNA- or DNA molecule, which may contain at least one modified nucleotide analog, i.e. a naturally occurring ribonucleotide or deoxyribonucleotide is substituted by a non-naturally occurring nucleotide. The modified nucleotide analog may be located for example at the 5′-end and/or the 3′-end of the nucleic acid molecule.

Nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase, such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine may be suitable. In sugar-modified ribonucleotides the 2′-OH-group is replaced by a group selected from H, OR, R, halo, SH, SR, NH 2, NHR, NR 2 or CN, wherein R is C 1-C 6 alkyl, alkenyl or alkynyl and halo is F, Cl, Br or I. In preferred backbone-modified ribonucleotides the phosphoester group connecting to adjacent ribonucleotides is replaced by a modified group, e.g. of phosphothioate group. It should be noted that the above modifications may be combined.

### Therapeutic Methods

An aspect of the present invention relates to the treatment of diseases characterized by the up-regulation and/or down-regulation of miRNAs. In one aspect, the present invention provides a method of treating a subject of a disorder associated with glucose mediated cell damage characterized in that said method comprises administering to the subject an agent or a mixture of agents that modulates the expression of one or more miRNAs in a cell or cells of the subject in need thereof.

In one embodiment, such treatments may comprise administering an agent or mixture of agents in order to down-regulate miRNAs and/or up-regulate the targets of said miRNAs.

In one embodiment of the present invention relates to treatments counteracting the up-regulation of miR144 or miR450 or both miR144 and miR450 in a cell or cells in need thereof, such as in glucose-mediated damaged cell or cells. It is contemplated that the results herein described for murine or rat miRNAs may be applied to the human counterpart miRNA. Such treatment may comprise the administration of inhibitory agents e.g. anti-sense molecules, to directly interact with the over-expressed miRNAs.

In one embodiment, the present invention relates to the treatment of diseases characterized by the down-regulation of miRNAs. Such treatments may comprise administering one or a mixture of miRNA modulators of the present invention in order to up-regulate the miRNAs and/or down-regulate the targets of said miRNAs. One embodiment of the present invention relates to treatments counteracting the down-regulation of one or more of miR1, miR146a, miR200b or miR320 in a glucose-mediated damaged cell or cells.

It is further contemplated the miRNA molecules described herein as down-regulated and/or down-regulated may similarly be used in treatment, diagnosis or screening methods. Such treatment may comprise the administration of at least one miRNA molecule (i.e. one or a mixture of miRNA molecules) to supplement the lack of one or more miRNAs or inducer of the expression of said one or more miRNAs.

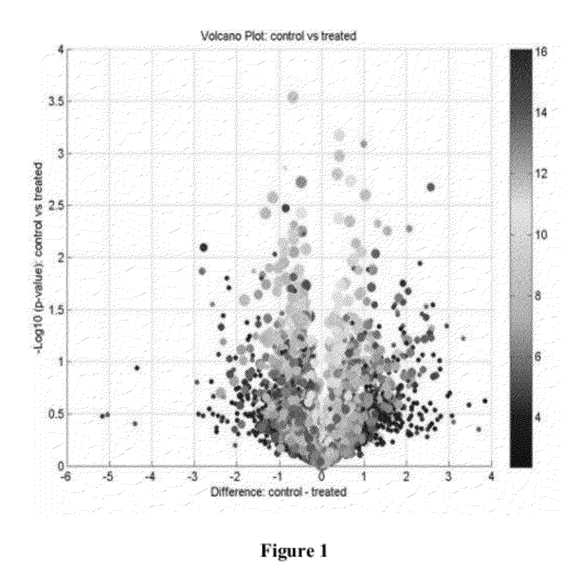

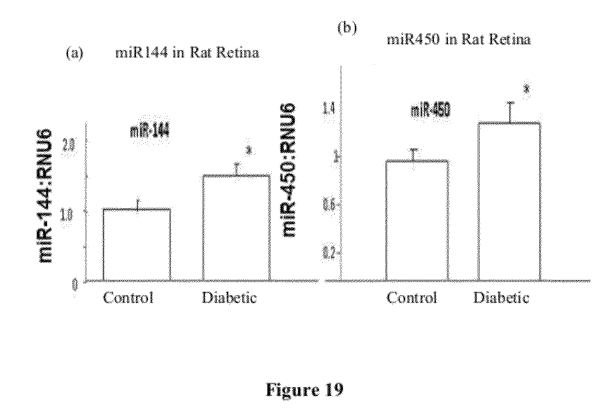

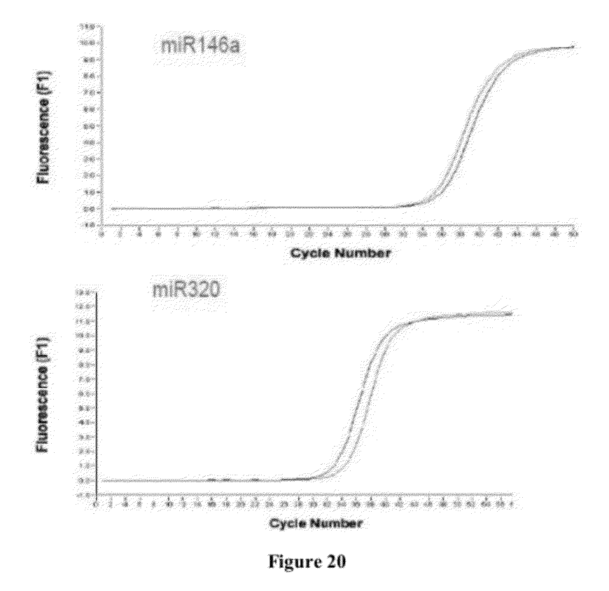

With reference to FIGS. 1-18 and 20, the Applicants discovered that miRNAs such as miR1, miR146a, miR200b, and miR320 may be down regulated in endothelial cells exposed to relatively high glucose levels, as well as in the retina of diabetic mammalian subjects. The Applicants further discovered that at least miR144 and miR450 may be up-regulated in the retina of diabetic subjects (see FIG. 19).

As such, agents that may modulate up-regulation of one or more of miR1, miR146a, miR200b, or miR320, and agents that may modulate down-regulation of one or more of miR144 or miR450 may be used in a method of treating a disorder associated with glucose mediated cell damage. In aspects of the present invention the disorder associated with glucose mediated cell damage may include a chronic diabetic complication, including diabetic retinopathy, diabetic nephropathy, or diabetic large vessels disease.

For example, miR200b, which, among others, targets translation of the vascular endothelial growth factor, a known regulator of angiogenesis, may function as a suppressor of angiogenesis. As vascular endothelial growth factor is also responsible to cause macular edema, miR200b may function as a suppressor of increased vascular permeability and edems. As previously stated, the proliferative stage of diabetic retinopathy may be characterized by progressive microvascular abnormalities such as angiogenesis. Thus expression or delivery of these agents, including miRNAs, or analogs, or precursors, or inhibitors thereof to cells or tissues may provide preventive and therapeutic efficacy, particularly against diabetic retinopathy.

It is contemplated that the therapeutic methods of the present invention may be used in combination with another method of treating a disorder associated with glucose cell damage.

For diagnostic or therapeutic applications, the miRNA or miRNA modulators may be included in a composition, such as a pharmaceutical composition. The pharmaceutical composition comprises as an active agent at least one of a miRNA or a miRNA modulator and optionally a pharmaceutically acceptable carrier.

### Delivery Vehicle

The administration of oligonucleotides of the present invention may be carried out by known methods, wherein a nucleic acid is introduced into a desired target cell in vitro or in vivo.

An aspect of the present invention comprises a nucleic acid construct comprised within a delivery vehicle. A, delivery vehicle is an entity whereby a nucleotide sequence can be transported from at least one media to another. Delivery vehicles may be generally used for expression of the sequences encoded within the nucleic acid construct and/or for the intracellular delivery of the construct. It is within the scope of the present invention that the delivery vehicle may be a vehicle selected from the group of RNA based vehicles, DNA based vehicles/vectors, lipid based vehicles, virally based vehicles and cell based vehicles. Examples of such delivery vehicles include: biodegradable polymer microspheres, lipid based formulations such as liposome carriers, coating the construct onto colloidal gold particles, lipopolysaccharides, polypeptides, polysaccharides, pegylation of viral vehicles.

In one embodiment of the present invention may comprise a virus as a delivery vehicle, where the virus may be selected from: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

Commonly used gene transfer techniques include calcium phosphate, DEAE-dextran, transfection, electroporation and microinjection and viral methods [12, 13, 14, 15, 16]. Another technique for the introduction of DNA into cells is the use of cationic liposomes [17]. Commercially available cationic lipid formulations are e.g. Tfx 50 (Promega) or Lipofectamin 2000 (Life Technologies).

The compositions of the present invention may be in form of a solution, e.g. an injectable solution, a cream, ointment, tablet, suspension or the like. The composition may be administered in any suitable way, e.g. by injection, particularly by intraocular injection, by oral, topical, nasal, rectal application etc. The carrier may be any suitable pharmaceutical carrier. Preferably, a carrier is used, which is capable of increasing the efficacy of the RNA molecules to enter the target-cells. Suitable examples of such carriers are liposomes, particularly cationic liposomes.

An aspect of the present invention further encompasses pharmaceutical compositions comprising one or more miRNAs or miRNA modulators for administration to subjects in a biologically compatible form suitable for administration in vivo. The administration of the miRNA modulators of the invention may act to decrease the production of one or more proteins that are overproduced in patients having diabetic retinopathy and/or to increase the production of one or more proteins that are under-produced in those patients, and thus reduce the glucose- and/or diabetic-related damage over time. The miRNAs of the invention may be provided within expression vectors as described above that are formulated in a suitable pharmaceutical composition.

By “biologically compatible form suitable for administration in vivo” is meant a form of the substance to be administered in which any toxic effects are outweighed by the therapeutic effects. Administration of a therapeutically active amount of the pharmaceutical compositions of the present invention, or an “effective amount”, is defined as an amount effective at dosages and for periods of time, necessary to achieve the desired result of increasing/decreasing the production of proteins. A therapeutically effective amount of a substance may vary according to factors such as the disease state/health, age, sex, and weight of the recipient, and the inherent ability of the particular polypeptide, nucleic acid coding therefore, or recombinant virus to elicit the desired response. Dosage regimen may be adjusted to provide the optimum therapeutic response. For example, several divided doses may be administered daily or at periodic intervals, and/or the dose may be proportionally reduced as indicated by the exigencies of the therapeutic situation. The amount of miRNA or miRNA modulator for administration will depend on the route of administration, time of administration and varied in accordance with individual subject responses. Suitable administration routes are intramuscular injections, subcutaneous injections, intravenous injections or intraperitoneal injections, oral and intranasal administration. In the case of diabetic retinopathy, injecting the miRNA- and/or miRNA modulator-based composition into the retina of the subject may be preferred. The composition of the invention may also be provided via implants, which can be used for slow release of the composition over time.

In the case of diabetic retinopathy, the miRNA- or miRNA modulator-based compositions of the invention may be administered topically to the eye in effective volumes of from about 5 microliters to about 75 microliters, for example from about 7 microliters to about 50 microliters, preferably from about 10 microliters to about 30 microliters. The miRNAs of the invention may be highly soluble in aqueous solutions. Topical instillation in the eye of miRNA in volumes greater than 75 microliters may result in loss of miRNA from the eye through spillage and drainage. Thus, it may be preferable to administer a high concentration of miRNA (e.g., 100-1000 nM) by topical instillation to the eye in volumes of from about 5 microliters to about 75 microliters.

In one aspect, the parenteral administration route may be intraocular administration. Intraocular administration of the present miRNA-based composition can be accomplished by injection or direct (e.g., topical) administration to the eye, as long as the administration route allows the miRNA modulators to enter the eye. In addition to the topical routes of administration to the eye described above, suitable intraocular routes of administration include intravitreal, intraretinal, subretinal, subtenon, peri- and retro-orbital, trans-corneal and trans-scleral administration. Such intraocular administration routes are within the skill in the art [18-21].

### Diagnostic Methods

In one embodiment, the present invention may also relate to diagnostic applications that take advantage of the different expression profiles of certain miRNAs in disorders associated with glucose-mediated cell damage, including a chronic diabetic complication, compared to a known normal standard. For example, the presence or absence of miRNAs may be tested in biological samples, e.g. in tissue sections or fluids such as blood, in order to determine and classify certain cell types, or tissue types, or miRNA-associated pathogenic disorders which are characterized by differential expression of miRNA-molecules or miRNA-molecule patterns. Further, the developmental stage of cells may be classified by determining temporarily expressed miRNA-molecules.

As such in one embodiment the present invention provides for a method for diagnosing a disorder in a subject, said disorder associated with glucose mediated cell damage. The method for diagnosing of the present invention may comprises measuring an expression profile of one or more miRNAs in a sample from the subject, wherein a difference in the miRNA expression profile of the sample from the subject and the miRNA expression profile of a normal sample or a reference sample may be indicative of the disorder associated with glucose mediated cell damage. The one or more miRNAs may be selected from miR1, miR146a, miR200b, miR320, miR144 or miR450.

The disorder associated with glucose mediated cell damage may comprise a chronic diabetic complication, including diabetic retinopathy, diabetic nephropathy, or diabetic large vessels disease

In one aspect the method for diagnosing of the present invention may be a method of diagnosing diabetic retinopathy in the subject.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

### Example 1

### 1. Material and Methods

### Animals

C57BL/6J mice were obtained from Jackson laboratories (Bar Harbor, Me., USA). Beginning at 6 weeks of age, the mice were randomly divided in two groups. One group of male mice were made diabetic by intraperitoneal injection of STZ [three 50 mg/kg consecutive injection on alternate days in citrate buffer, pH=5.6]. Sex-matched littermates were used as controls and were given an equal volume of citrate buffer. Diabetes was defined as blood glucose level >20 mmol/L on two consecutive days (Freestyle Mini, TheraSense Inc., Alameda, Calif., USA). The animals were fed on a standard rodent diet and water ad libitum and were monitored for hyperglycemia, glucosuria and ketonuria (Uriscan Gluketo™, Yeong Dong Co., Seoul, South Korea) [22-25]. None of the animals received exogenous insulin. The animals (n=6 per group) were sacrificed after 2 months of diabetes. Retinal tissues were dissected out and snap frozen.

db/db mice (a model for type 2 diabetes mellitus) and their control mice were purchased from Jackson laboratories. Following onset of diabetes (blood glucose estimation), they were followed up for a period of two months. Metabolic parameters, body weight, urine sugar, urine ketones were monitored for two months. At the end of this period, the mice were sacrificed and retinal tissues collected. miRNA were extracted and analysed (see below).

Male Sprague-Dawley rats (200-250 g) were obtained from Charles River Colony and were randomly divided into control and diabetic groups. Methods of diabetes induction and monitoring have previously been described [22-24]. After 4 weeks, the animals (n=6/group) were sacrificed and the retinal tissues were snap-frozen for gene expression and microRNA analysis or placed in 10% formalin for paraffin embedding.

### Human Tissue

5 μm retinal tissue sections from the formalin fixed paraffin embedded tissue were collected on the positively charged slides from the surgically removed eyes from the archives of London Health Sciences Centre. Ethics approval was obtained before such collection. Such materials were collected from both non-diabetic and diabetic individuals who underwent enucleation for untreated conditions. The sections were stained for albumin to test for permeability and LNA™ ISH for miR200b localization (See below).

### Endothelial Cells

Human umbilical vein endothelial cells (HUVECs; American Type Culture Collection, Rockville Md.), which show glucose-induced abnormalities, were plated at 2,500 cells/cm2 in endothelial growth medium (EGM)(Clonetics, Rockland, Me.). EGM was supplement with 10 μg/l human recombinant epidermal growth factor, 1.0 mg/l hydrocortisone, 50 mg/l gentamicin, 50 μg/l amphotericin B, 12 mg/l bovine brain extract, and 10% fetal bovine serum. Appropriate concentrations of glucose were added to the medium when the cells were 80% confluent. All experiments were carried out after 24 h of glucose incubation unless otherwise indicated. The inhibitors were added 30 minutes before addition of glucose. At least, three different batches of cells, each in triplicate, were used for each experiment. Cell viability and proliferation were determined by 2-(4-Iodophenyl)-3-(4-nitrophenyl)-5-(2,4-disulfophenyl)-2H-tetrazolium (WST-1; Roche, Laval, PQ). This colorimetric assay for the quantification of cell proliferation and cell viability, is based on the cleavage of the tetrazolium salt WST-1 by mitochondrial dehydrogenases. Briefly, the HUVECs were seeded onto 96-well plates at a density of 1.0×104 cells per well in 100 μl culture medium with or without incubation with specific reagents for 24 hours. Ten μl of WST-1 was added per well and the cells were incubated for 4 hours at 37° C. The absorbance at 450 nm was measured [26-28].

Bovine retinal capillary endothelial cells (BRECs) were obtained from VEC technologies (Rensselaer, N.Y.) and were grown in the fibronectin-coated flask in a defined EC growth medium (MCDB-131 complete, VEC Technologie). Before transfection 24 hours, the cells were passaged in the 6 well plate coated with fibronectin (Sigma, USA). The culture conditions have previously been described by others [29].

HEK293A cells were also obtained from ATCC and were used as previously described by others [30]. All cell culture experiments were performed in triplicate for 4 times or more. All reagents were obtained from Sigma Chemicals (Sigma, Oakville, Ontario, Canada) unless otherwise specified.

Microarray Analysis for miRNA Expression

MicroRNAs were extracted from endothelial cells and retinal tissues using the mirVana miRNA isolation kit (Ambion Inc., Austin, Tex., USA). Briefly, the tissues were homogenized in the Lysis/Binding solution. miRNA additive (1:10) and equal volume acid-phenol:chloroform was added to the lysate and incubated for 10 min on ice. Following centrifugation and removal of the aqueous phase, the mixture was incubated in ethanol. The mixture was passed through the filter cartridge and was eluted with elution solution.

Retinal miRNA arrays were custom analyzed using services from Asuragen. Such analyses were performed using Agilent miRNA Arrays (http://assuragen.com).

PCR-based miRNA array analyses were carried out to examine alteration of miRNAs expression in human umbilical vein endothelial cells (HUVEC), exposed to glucose using TaqMan™ PCR system according to the manufacturer's instructions.

miRNA Analysis by RT-PCR

miRNAs were isolated using mirVana kit (Ambion Inc., Austin, Tex., USA). Real-time PCR was used to validate array data. The primers (Table 2) were obtained from Ambion Inc. (Austin, Tex., USA). For a final reaction volume of 20 μL, the following reagents were added: 10 μL TaqMan 2× Universal PCR Master Mix (No AmpErase UNG), 8 μL Nuclease-free water, 1 μL TaqMan microRNA probe and 1 μL cDNA product. The data was normalized to RNU6B to account for differences in reverse-transcription efficiencies and amount of template in the reaction mixtures.

### Western Blotting

Endothelial cells and retinal tissues were homogenized, centrifuged and resuspended in ice cold RIPA lysis buffer (0.5 M Tris-HCl, pH 7.4, 1.5 M NaCl, 2.5% deoxycholic acid, 10% NP-40, 10 mM EDTA). Cell lysates were subsequently sonicated and the protein was quantified by BCA assay (Pierce Endogen, IL, USA). To confirm expression of MAPK (ERK1/2) (New England Biolab), 30 μg of protein was loaded and size-fractionated on a 10% SDS-PAGE and blotted overnight onto a PVDF membrane (BIO-RAD Hercules, Calif., USA). Nonspecific sites were blocked in a 5% solution of nonfat milk powder in TBS. This was followed by incubation with goat anti-rabbit secondary IgG antibody with horseradish peroxidase conjugate (Santa Cruz Biotechnology Inc., CA, USA) using 1:1000 and 1:5000 dilutions, respectively. Lysates containing MEF2 were visualized with enhanced chemiluminescence Advance Western blot detection system (Amersham Biosciences, Piscataway, N.J., USA) and Alphaimager 2200. β-actin expression was tested using the same membrane as an internal control.

miRNA Mimic Transfection

The endothelial cells were transfected with miRIDIAN™ micro RNA mimics of miR146a, miR200b, and miR320 (20 nM) (DHARMACON Inc., Chicago, Ill., USA) using transfection reagent (Qiagen, ON, Canada). The constructs were custom synthesized from Dharmacon Inc. Related oligonucleotide sequences, based on C. elegans miRNA with similar design and modifications as miRIDIAN microRNA, mimics, were used as negative control miRNA (20 nM) for control transfection (also referred to in this document as negative transfection). miRNA transfection efficiency was determined by realtime RT-PCR.

For retinal transfection: diabetes was induced in the male Sprague Dawley (SD) rats using streptozotocin (STZ, 65 mg/kg, in citrate buffer IP, controls received buffer only). Diabetes was defined as blood glucose level >20 mmol/L on two consecutive days and was confirmed by testing of blood glucose. For the treatment of the animals, 1.4 μg miR146a or miR200b in transfection reagent (a total volume of 10 μl) were injected into the right vitreous cavity of each rat, once a week for four weeks. Similar volume of control miRNA (non-specific microRNA without any specific binding) in transfection reagent was injected in the left eye of the animal. The animals were sacrificed and retinal tissues were collected one week after the last injection. Retinal tissues were dissected out and were used to extract total RNA and miRNA for the real time RT-PCR or microRNA analysis.

Control rats were injected with the same volume of saline and Lipofectin Reagents. Custom miRmimics or antigomirs were synthesized by Dharmacon based on mature microRNA sequences of hsa-miR200b (SEQ ID NO. 3) and scrambled control 5′UCACAACCUCCUAGAAAGAGUAGA3′; SEQ ID NO. 7). Intravitreal p300 siRNA injection has previously been described [31]. The animals were sacrificed in 5^{th} week and the retinal tissues were collected as above.

### Cell Viability

Cell viability was examined by trypan blue dye exclusion test. Trypan blue stain was prepared fresh as a 0.4% solution in 0.9% sodium chloride. The cells were washed in PBS, trypsinized, and centrifuged. Twenty microlitres of cell suspension were added to 20 μL of trypan blue solution and 500 cells were microscopically counted in Burker cytometer. Cell viability was expressed as a percentage of the trypan blue negative cells in untreated controls [26-28].

### Luciferase Assay

a) VEFG: The 3′UTRs of VEFG from rat and human genomes were used with carried Sac I and Hind III restriction site in the forward and reverse position, respectively. The primers for human VEGF 3′UTR cloning are listed below. The amplicons from 3′UTRs and the complementary sequences were cloned into pCR®2.1 vector and amplified in DH5a competent cells (Invitrogen, Burlington, ON, Canada) and confirmed by sequencing. The target gene insert was then subcloned into the pMIR-REPORT™ vector (Ambion, Austin, Tex., USA) and amplified in DH5a competent cells (Invitrogen) and confirmed by sequencing. The pMIR_VEGF 3′UTR, a miRNA mimic and pMIR-Report β-gal control plasmid were then cotransfected into the 293A cells. Nucleotide substitutions were introduced by PCR mutagenesis to yield mutated binding site. Forty-eight hours after transfection, luciferase activity was measured using the Dual-Light Chemiluminescent Reporter Gene Assay System (Applied Biosystems) following the manufacturer's instructions. Luciferase activity was read using Chemiluminescent SpectraMax M5 (Molecular Devices, Sunnyvale, Calif.). Luciferase activity was normalized for transfection efficiency by measuring β-galactosidase control activity according to the manufacturer's instructions. The experiments were performed in triplicate [30].

The primers for human and rat VEGF 3′UTR mutation cloning are:

Human VEGF 5′
Forward primer:
(SEQ ID NO. 8)
5′AGAGCTCCCCGGCGAAGAGAAGAGAC 3′
Reverse primer,
(SEQ ID NO. 9)
TCTAGAAAGCTTGGAGGGCAGAGCTGAGTGTTA 3′
Rat VEGF
Forward primer
(SEQ ID NO. 10)
5′ AGAGCTCGGGTCCTGGCAAAGAGAAG 3′
Reverse primer
(SEQ ID NO. 11)
5′ TCAAGCTTGGAGGGCAGAGCTGAGTGTTA 3′

b) Fibronectin (FN): FN1 3′-UTR and antisense sequence of miR146a (or scrambled control) were cotransfected in the 293A cells with the pMIR REPORT Luciferase vector (vector) and pMIR reporter control vector containing B-gal with CMV promoter. Forty-eight hours after transfections, cell extracts were assayed for luciferase expression. Relative promoter activities were expressed as luminescence units normalized for β-galactosidase expression.

For luciferase reporter experiments, a human fibronectin (FN) 3′-UTR segment of 617 bp and a rat FN 3′-UTR segment of 644 bp were amplified by PCR from human and rat cDNA and inserted into the pMIR REPORT Luciferase vector with CMV promoter (Applied Biosystems Inc, CA, USA) by using the Sac I and Hind III sites immediately downstream from the stop codon of luciferase. The pMIR_FN 3′UTR, miR-200b mimic (or scrambled) and pMIR REPORT Luciferase vector reporter control vector containing B-gal with CMV promoter were then cotransfected into the 293A cells Forty-eight hours after transfection, luciferase activity was measured using the Dual-Light Chemiluminescent Reporter Gene Assay System (Applied Biosystems) following the manufacturer's instructions. Luciferase activity was read using Chemiluminescent SpectraMax M5 (Molecular Devices, Sunnyvale, Calif.). Luciferase activity was normalized for transfection efficiency by measuring β-galactosidase control activity according to the manufacturer's instructions. The experiments were performed in triplicate [30].

The following sets of primers were used to generate specific fragments:

Human FN 3′-UTR,
Forward primer,
(SEQ ID NO. 12)
5′-AGAGCTCATCATCTTTCCAATCCAGAGGAAC-3′;
Reverse primer,
(SEQ ID NO. 13)
5′-TCAAGCTTTAATCACCCACCATAATTATACC-3′;
Rat FN 3′-utr,
Forward primer,
(SEQ ID NO. 14)
5′-AGAGCTCTCCAGCCCAAGCCAACAAGTG-3′;
Reverse primer,
(SEQ ID NO. 15)
5′-TCAAGCTTTCCACAGTAGTAAAGTGTTGGC-3′

Underlined sequences indicate the endonuclease restriction site.

### RNA Extraction and RT-PCR

RNA was extracted with TRIzol™ reagent (Invitrogen Canada Inc., ON, Canada) as previously described [26-28,31]. Total RNA (2 μg) was used for cDNA synthesis with oligo (dT) primers (Invitrogen Canada Inc., ON, Canada). Reverse transcription was carried out by the addition of Superscript™ reverse transcriptase (Invitrogen Canada Inc., ON, Canada). The resulting cDNA products were stored at −20° C. Real-time quantitative RT-PCR was performed using the LightCycler (Roche Diagnostics Canada, QC, Canada). For a final reaction volume of 20 the following reagents were added: 10 μL SYBR™ Green Taq ReadyMix (Sigma-Aldrich, ON, Canada), 1.6 μL 25 mmol/L MgCl2, 1 μL of each forward and reverse primers (Table 2), 5.4 μL H₂O, and 1 μL cDNA. Melting curve analysis was used to determine melting temperature (Tm) of specific amplification products and primer dimers. For each gene, the specific Tm values were used for the signal acquisition step (2-3° C. below Tm). The data was normalized to 18S RNA or β-actin mRNA to account for differences in reverse-transcription efficiencies and amount of template in the reaction mixtures.

TABLE 2
Oligonucleotide sequences for RT-PCR

| Gene | Sequence | |
|---|---|---|
| ANP | 5′ CTGCTAGACCACCTGGAGGA 3′ | SEQ ID NO 16 |
| | 5′ AAGCTGTTGCAGCCTAGTCC 3′ | SEQ ID NO 17 |
| BNP | 5′ GACGGGCTGAGGTTGTTTTA 3′ | SEQ ID NO 18 |
| | 5′ ACTGTGGCAAGTTTGTGCTG 3′ | SEQ ID NO 19 |
| 18S rRNA | 5′ GTAACCCGTTGAACCCCATT 3′ | SEQ ID NO 20 |
| | 5′ CCATCCAACGGTAGTAGCG 3′ | SEQ ID NO 21 |
| β-actin | 5′ CATCGTACTCCTGCTTGCTG 3′ | SEQ ID NO 22 |
| | 5′ CCTCTATGCCAACACAGTGC 3′ | SEQ ID NO 23 |
| ET-1 | 5′ AAGCCCTCCAGAGAGCGTTAT 3′ | SEQ ID NO 24 |
| | 5′ CCGAAGGTCTGTCACCAATGT 3′ | SEQ ID NO 25 |
| VEGF | 5′ GGCCTCCGAAACCATGAACTTTCT | SEQ ID NO 26 |
| | GCT 3′ | |
| | 5-GCATGCCCTCCTGCCCGGCTCACCG | SEQ ID NO 27 |
| | C 3′ | |
| FN | 5′ GATAAATCAACAGTGGGAGC 3′ | SEQ ID NO 28 |
| | 5′ CCCAGATCATGGAGTCTTTA 3′ | SEQ ID NO 29 |

Regarding ET-1 SEQ ID NO 24: minor groove-binding probes were used (Taqman; Applied Biosystems, Foster City, Calif.) to avoid signal acquisition from nonspecific amplification products. These probes are modified at the 5′ end by the addition of 6-carboxyfluorescein (FAM) and at the 3′ end by the addition of a nonfluorescent quencher (MGBNFQ). As elongation proceeds, FAM is cleaved by the exonuclease activity of DNA Taq polymerase and an increase in reporter fluorescence emission takes place. The reporter dye (FAM, Taqman, Applied Biosystems) exhibits excitation and emission in the same range as SYBR I, which allows detection with the same detector channel. Hence there is an extra sequence for this ET-1 probe with FAM at one end and MGBFNQ at other end.

### ELISA

ELISA for VEGF was performed using a commercially available kit for human and rat VEGF (ALPCO, Salem, N.H., USA; R&D Systems, Minneapolis, Minn., USA) according to the manufacturer's instructions [26].

### Permeability Assay

HUVECs were seeded onto inserts (1 μm pores) in 24-well plates with or without incubation with specific reagents for 24 hours, and were tested for vascular permeability using the In Vitro Vascular Permeability Assay Kit (Millipore, Billerica, Mass., USA) according to the manufacturer's instructions [32].

### Angiogenesis Assay

An in vitro Angiogenesis Assay Kit (Chemicon, Billerica, Mass., USA) was used to evaluate tube formation of HUVECs. Tube formation was quantified using branch point counting using Infinity Capture Application Version 3.5.1 on Leica Microsystems inverted microscope [33].

### Immunohistochemistry

Rat and human retinal sections were immunocytochemically stained for albumin to examine for increased vascular permeability using anti-human albumin antibody (1:500) (Abcam, Inc, Cambridge, Mass., USA). These methods have previously been described [25].

### In Situ Hybridization

Rat and human retinal sections were labelled for miR200b expression. Five micrometer thick retinal tissue sections from formalin-fixed, paraffin-embedded blocks were transferred to positively charged slides to be used for labelling. A 5′ and 3′ double DIG-labelled custom-made mercury LNA™ miRNA detection probes (Exiqon, Vedbaek, Denmark) were used to detect miR200b expression along with the In Situ Hybridization (ISH) Kit (Biochain Institute, Hayward, Calif., USA) [34].

### Statistical Analysis

All experimental data are expressed as mean±SD and were analysed by ANOVA and post-hoc analysis or by t-test as appropriate. A p value of 0.05 or less was considered significant.

### 2. Results

a. MicroRNA (miRNA) Array Analysis of Diabetic Rat Retina
Diabetes Causes miRNA Alterations in the Retina:

Working under the hypothesis that the expression of key genes in diabetes may, in part, be regulated by miRNAs, the Applicants first searched for miRNAs whose expression changed in diabetes. To this end, the Applicants used an animal model of chronic diabetes. Streprotozotocin (STZ)-induced diabetic rats exhibit molecular and early structural and functional changes of DR [32-34]. To examine miRNA alteration in DR, microarray analysis was carried out on the retinal tissues from male STZ induced diabetic rats after 1 month of diabetes, and on age- and sex-matched controls. Diabetic animals showed hyperglycemia (serum glucose of diabetics 19.2±4.7 mmol/L vs. controls 7.0±0.8 m.mol/L, P<0.005) and reduced body weight (body weight of diabetics 372.0±34.7 g. vs. controls 445.7±17.4 g. P<0.01). Microarray analyses of miRNAs extracted from these tissues was performed (FIG. 1). Such analyses showed alterations of multiple miRNAs in the retina of these animals (FIG. 1). Using open sourced softwares (www.TargetScan.org, www.microrna.org, www.ebi.ac.uk1) for miRNA target predictions, miRNAs associated with known genes/proteins that are altered in DR were identified.

FIG. 1 illustrates a miRNA array-volcano plot, showing miRNA alteration in control vs. treated (diabetic) rat retina.

Each circle of FIG. 1 represents one miRNA. The size of the circle for each probe is proportional to the miRNA detection rate for the entire experiment, with larger spots representing a higher % present. The circles are colored according to the average expression of the probe across the two groups, according to the grey scale provided on the right of the plot. Circles to the left of −1, difference line and to the right of the 1 difference line are considered to have a fold change >2× (x-axis is the log 2 of the fold-change between two experimental groups). miRNAs of interest, miR1, miR146a, miR200b, and miR320, were down-regulated, whereas miR144 and miR450 were up-regulated in the retina of diabetic rats compared to controls [custom analysis using Asuragen miRNA system].

b. miR320 Expression Levels in HUVECs Exposed to High Glucose

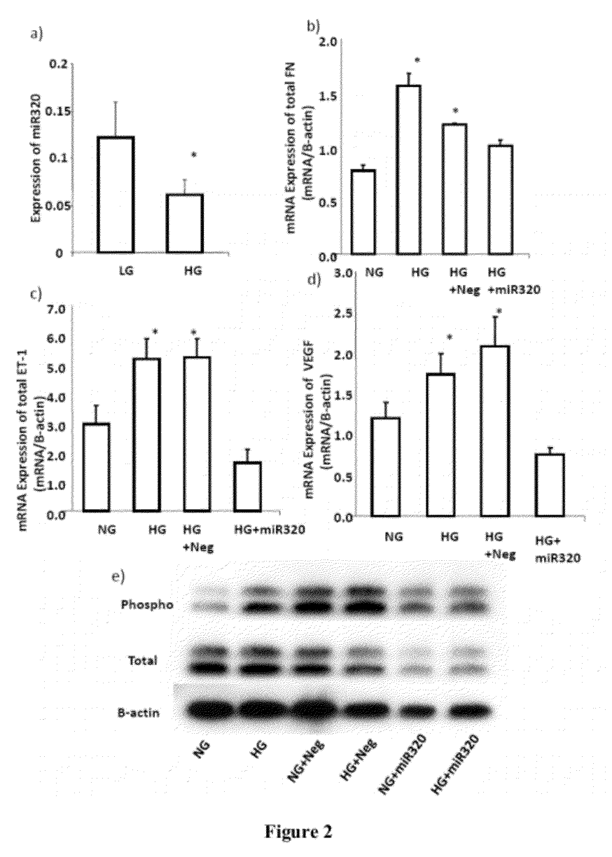

Real time quantitative polymerase chain reaction (qPCR) analysis of the expression levels of miR320 was studied in HUVECs exposed to high levels of glucose. Results are shown in FIG. 2. Panel a) of FIG. 2 shows a statistically significant decrease in the expression of miR320 in the endothelial cells exposed to 25 mM glucose (high glucose, HG) compared to endothelial cells exposed to 5 mM glucose (low glucose, LG). Such miR320 down-regulation was associated with a statistically significant up-regulation of fibronectin (FN, FIG. 2 b)), endothelin-1 (ET-1, FIG. 2 c)) and vascular endothelial growth factor (VEGF, FIG. 2 d)) mRNAs; as well as MAPK (ERK1/2) activation (FIG. 2 e)). miR320 mimic (miR320) transfection prevented such abnormal up-regulation, whereas negative transfection (Neg) was ineffective (FIG. 2, panels b)-e)). ERK activation is an important step in diabetic retinopathy [28]

c. miR146a Expression in Endothelial Cells Exposed to High Glucose and in Retina of Diabetic Rats

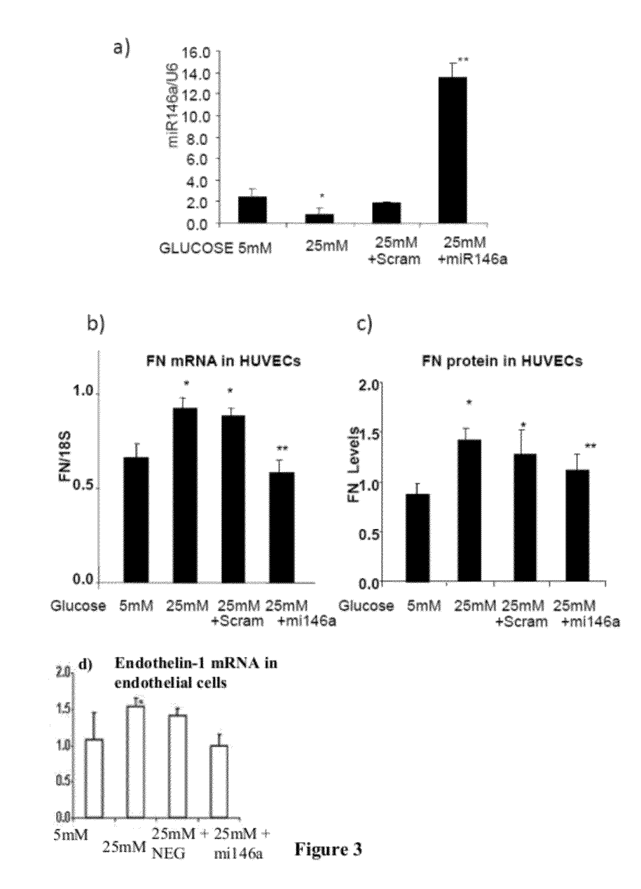

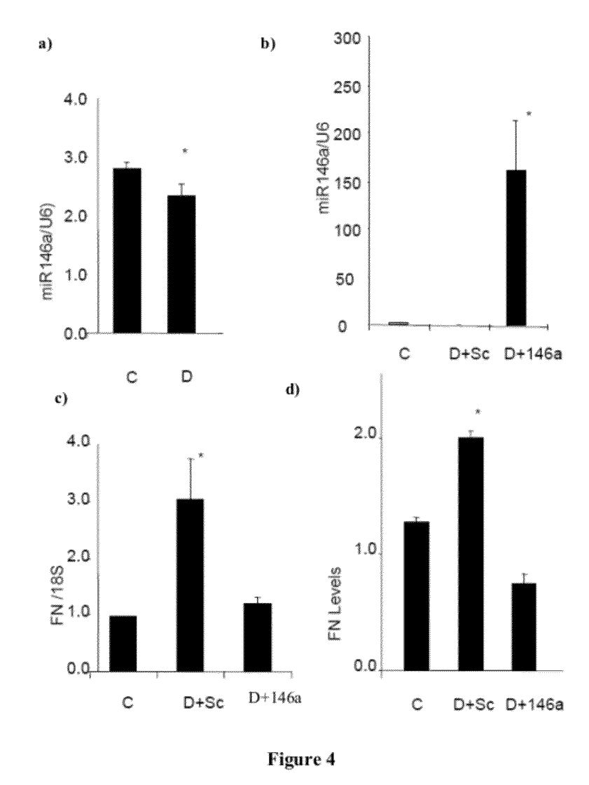

The down-regulation of miR146a in the retina of diabetic rats shown in FIG. 1 was verified with qRT-pCR. qRT-pCR analysis of the expression levels of miR146a was studied in HUVECs exposed to high levels of glucose (FIG. 3, a)) and in rat retina (FIG. 4, panels a) and b)). As shown in FIG. 3 a) miRNA 146a was down-regulated in the HUVECs when exposed to 25 mmol/L. glucose compared to 5 mmol./L glucose. FN mRNA (FIG. 3 b)) and FN protein (FIG. 3 c)) were down-regulated in the ECs when exposed to 25 mmol/L. glucose compared to 5 mmol./L glucose. Transfection of endothelial cells with miR146a mimics (but not the scrambled mimics) normalized HG induced down-regulation of miR146 (FIG. 3 a)), up-regulation of FN mRNA (FIG. 3 b)) and FN protein (FIG. 3 c)). miR146 mimic transfection of ECs (HG+mi146a) also prevented up-regulation of ET-1 mRNA (FIG. 3 d)), whereas negative transfection (HG+Neg) was ineffective (FIG. 3 d)) (see explanation for negative transfection above). In FIG. 3 a) efficiency of miR146a mimic transfection is also shown by increased miR146 expression in the HUVECs following miR146a mimic transfection compared to scrambled mimics.

Injecting miR146a mimics in the vitreous cavity of the eye of diabetic rats normalized diabetes induced up-regulation of miR146a and fibronectin (FN), one of the important molecules increased in the retina in diabetes (FIG. 4). Retinal tissues from the STZ induced diabetic rats: Diabetic rats (D) demonstrated reduced miR146a levels compared to age and sex matched controls (C) (FIG. 4 a)). In parallel, retinal tissues from the diabetic rats demonstrated up-regulation of FN mRNA (FIG. 4 c)) and protein (FIG. 4 d)). Diabetes induced FN mRNA and protein up-regulation were prevented by intravitreal injection of miR 146a mimic (D+miR146a) but not by scrambled controls (D+SC). FIG. 4 b) shows efficiency of intravitreal delivery in which intravitreal injection of miR146a mimic (but not scrambled mimic) lead to increased retinal miR146a [Data expressed as a ratio of A) RNU6, B) 18S, *=significantly different than corresponding C, n=5/group].

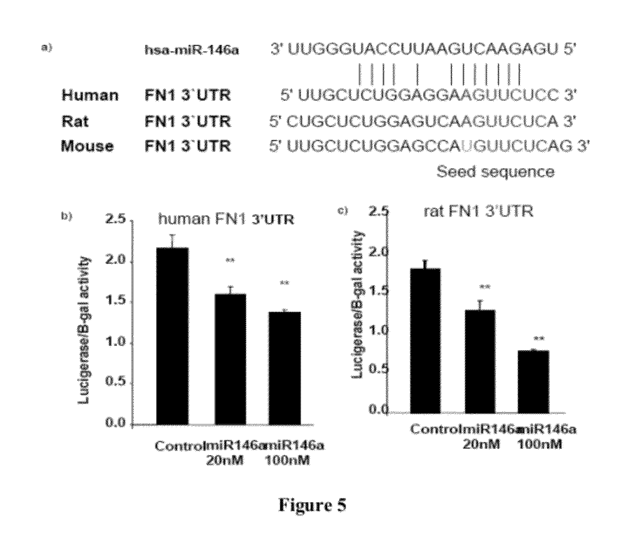

To further validate miR146a targeting of FN, the Applicants examined the binding of miR146a with 3′UTR of the FN1 gene. Luciferase reporters containing miR146a complimentary site from human and rat (in separate experiments) FN1 3′-UTR and antisense sequence of miR146a (or scrambled control) were co-transfected in HEK-293A cells with the pMIR REPORT Luciferase vector (vector) and pMIR reporter control vector containing B-gal with CMV promoter. 48 hours after transfections, cell extracts were assayed for luciferase expression. Relative promoter activities were expressed as luminescence units normalized for β-galactosidase expression. Alignment of FN 3′UTR sequence with mature miR146a was based on bioinformatics predictions (www.TargetScan.org, www.microrna.org, www.ebi.ac.uk1). The 5′ end of the mature miR146a is the seed sequence and demonstrates perfect complementarity with seven nucleotides of the 3′ UTR of FN (FIG. 5 a). Binding of miR146a with FN promoter Luciferase reporter assay shows dose dependent binding of FN 3′UTR with miR146a Luciferase reporters containing miR146a complimentary site from human (FIG. 5 b)) and rat (FIG. 5 c)).

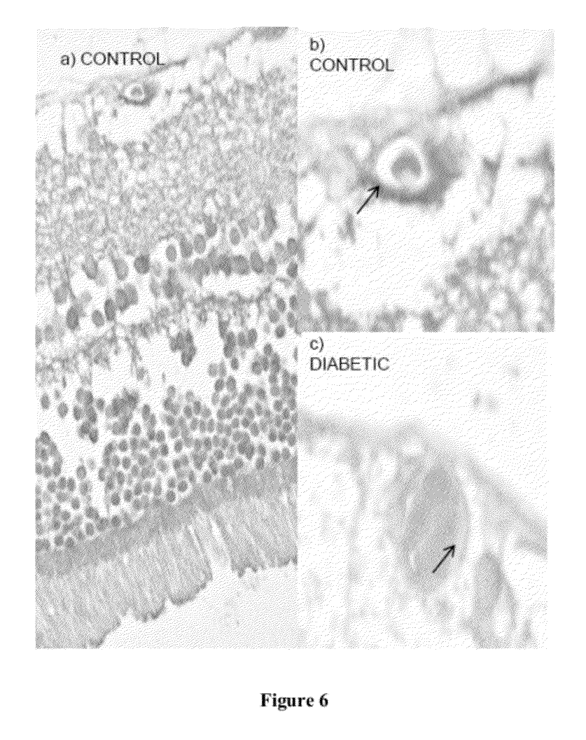

FIG. 6 a) is a micrograph of a LNA™-ISH study of retinal tissues in a control rat retina showing localization of miR146a. FIG. 6b) shows a higher magnification micrograph with positive staining for miR146a in the retinal capillaries (arrow). FIG. 6 c) is a micrograph of a LNA-ISH study of retinal tissues in a diabetic rat retina showing minimum (if any) expression of miR146a in the capillaries indicating diabetes induced vascular permeability (ALK Phos was used as chromogen with no counterstain).

miR146a Regulates Glucose Induced NFkB Activity

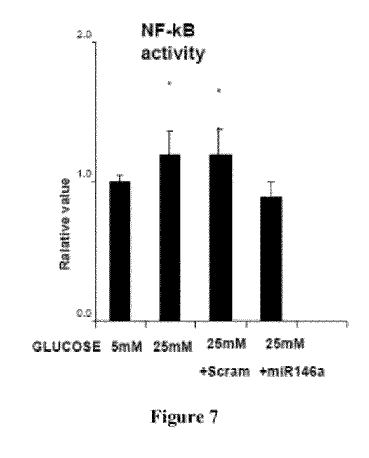

As illustrated in FIG. 7, miR146a mimic (miR146a) transfection prevented glucose induced NFkB activation in the ECs. NFkB activation is an important step in DR [27,31].

miR146a Down-Regulation is Present in Mice Diabetic Retinopathy (Model of Type 2 Diabetes)

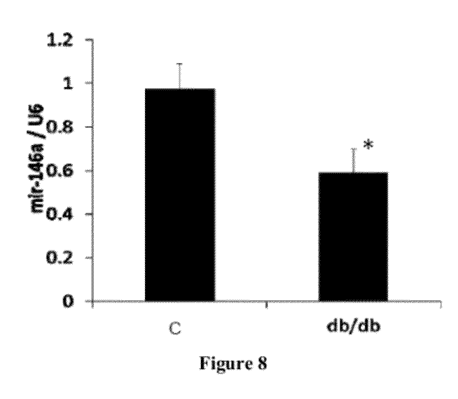

As illustrated in FIG. 8, the investigator further demonstrated by qRT-PCR analysis that miR146a levels in is statistically significant reduced in the retinal tissues from the db/db diabetic mice (db/db) (a model of type 2 diabetes) compared to miR146a levels in age and sex matched controls (C).

d. miR200b
Diabetes Causes miR200b Down-Regulation in the Retina

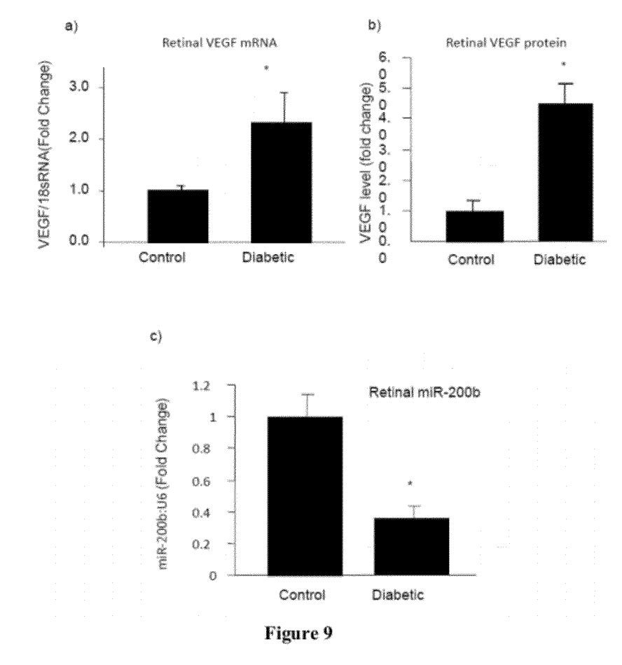

Down-regulation of miR200b in the retina of diabetic rats shown in FIG. 1 was verified with qRT-pCR (FIG. 9 c)). miR200b is a VEGF targeting miRNA. Retinal tissues of the diabetic rats showed increased levels of VEGF mRNA and protein as measured by qRT-PCR and ELISA (FIG. 9 a) and b)). Other members of miR200b cluster, namely miR429, was not significantly altered under diabetic conditions) and miR-200a does not target VEGF. Hence, an association was established between miR200b down-regulation and VEGF up-regulation in DR. To test the specificity of the association between miR200b and VEGF, the Applicants examined whether FN, another bioinformatics based target of miR200b and a protein of interest in DR, is regulated by miR200b. However, no direct regulation of FN by miR200b was observed (data not shown).

miR200b Regulates Glucose Induced VEGF Up-Regulation in the Endothelial Cells

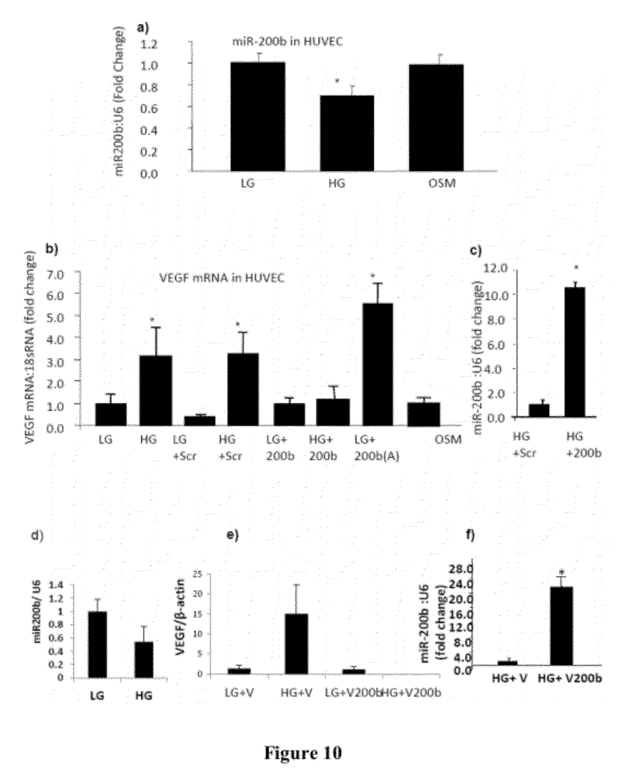

To establish a cause-effect relationship between miR200b and VEGF, Applicants first used an in vitro model system. As endothelial cells (ECs) are the primary cellular targets in DR, the Applicants used HUVECs in culture to study the mechanistic aspects and the functional significance of miR200b alterations. It has been shown that ECs exposed to high levels of glucose (simulating hyperglycemia) recapitulate molecular and functional features of diabetic vascular pathologies [2§-28]. The Applicants found that high levels of glucose cause changes in miR200b levels. 25 mmol/L D-glucose (HG) (compared to 5 m.mol/L D-glucose LG)) causes a significant down-regulation of miR200b (FIG. 10 a)). These levels of glucose were established using a dose-response analysis of VEGF expression (data not shown) and previous experiments by the Applicants and others [35, 30, 36]. No change in miR200b level was observed when the ECs were challenged with 25 m.mol/L L-glucose (FIG. 10 a), OSM). In parallel to decreased miR200b upon exposure to HG, mRNA and protein levels of VEGF (measured by qRT-PCR and ELISA) were increased. Such increases were prevented by miR200b mimics transfection. On the other hand, transfection of miR200b antigomir demonstrated gluco-mimetic effects by up-regulating VEGF transcripts (FIG. 10 b)).

To further establish a direct relevance of these findings in the context of diabetic retinopathy the Applicants examined whether similar changes occurs in the retinal capillary endothelial cells. The results show that 25 mmol/L D-glucose (HG) (compared to 5 m.mol/L D-glucose (LG)) causes a significant down-regulation of miR200b (FIG. 10 d)). In parallel VEGF mRNA was up-regulated following exposure to HG (FIG. 10 e). Transfection of miR200b mimics prevented glucose induced VEGF up-regulation (FIG. 10 e).

miR200b Regulates Glucose Induced Functional Alterations in the Endothelial Cells

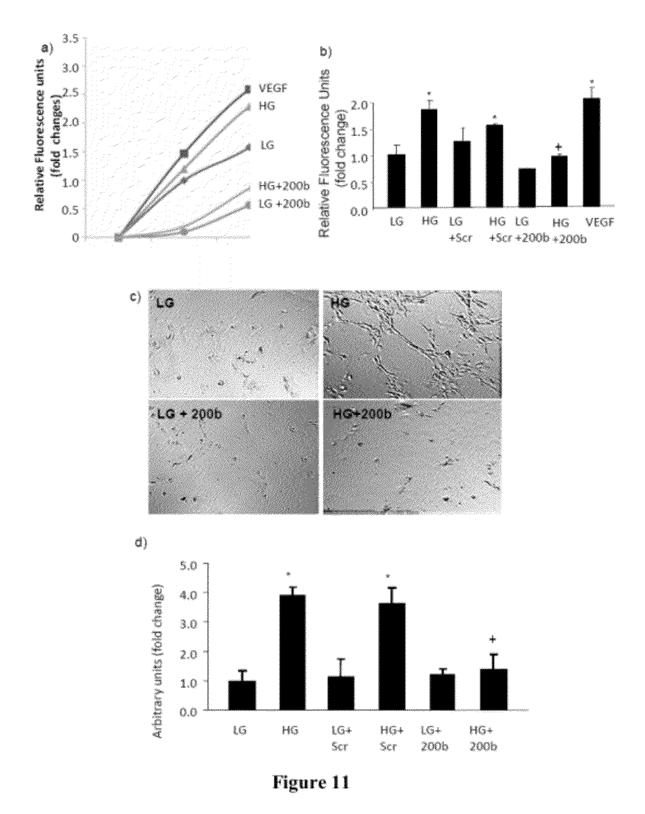

The Applicants next examined endothelial permeability and tube formation, two characteristic functional effects of VEGF in this system. HUVECs showed increased permeability and tube formation following treatment with HG and VEGF peptide (FIG. 11 a)-d)). To examine functional significance of miR200b, we transfected miR200b mimics (and scrambled controls) in HUVECs exposed to HG. Transfection efficiency was confirmed by analyzing the abundance of miR200b in these cells (FIG. 10c)). Upon transfection, we observed a normalized of glucose-induced up-regulation of VEGF as well as augmented HG-induced endothelial permeability and tube formation (FIG. 10 b)-c)) (FIG. 11 a)-d)). These results established a direct regulatory relationship between miR200b on HG-induced VEGF expression and its functional consequences.

To further validate miR200b targeting of VEGF, the Applicants examined the binding of miR200b with 3′UTR of the VEGF gene. Luciferase reporters containing miR200b complimentary site from human and rat (in separate experiments) VEGF 3′-UTR and antisense sequence of miR200b were co-transfected in HEK-293A cells.

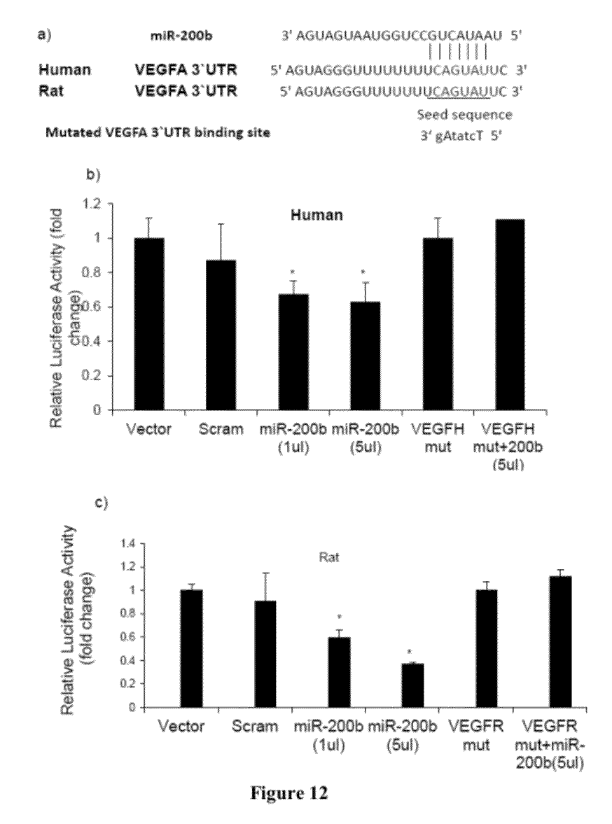

FIG. 12 a) illustrates the alignment of VEGF 3′UTR (and mutated VEGF3′-UTR) sequence with mature miR200b based on bioinformatics predictions (www.TargetScan.org, www.microrna.org, www.ebi.ac.uk1). The 5′ end of the mature miR200b is the seed sequence and has perfect complementarity with seven nucleotides of the 3′ UTR of VEGF. FIG. 12 b) (human), and FIG. 12 c) (rat) show that ectopic overexpression of miR200b significantly repressed VEGF 3′UTR luciferase activity, indicating a direct binding. No such effects were seen when VEGF mutated (VEGFH mut and VEGFR mut in FIG. 12 b) and c) respectively) was used.

miR200b is Present in the Retina and Regulates Diabetes Induced Retinal VEGF Up-Regulation

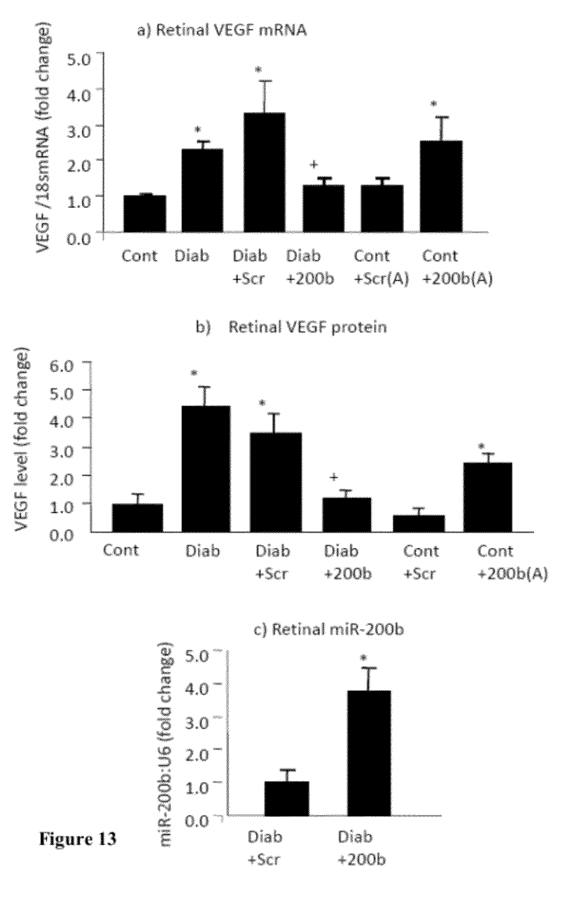

Having established VEGF targeting by miR200b in vitro, the Applicants then tested whether miR200b targets VEGF in the diabetic animal model. miR200b mimic was injected in the vitreous cavity of one eye of the diabetic rats at 1.4 μg/week for four weeks (the other eye received the same dose of scrambled control). In a separate set of experiments, the Applicants injected intravitreal miR200b antigomirs to non-diabetic, rats to produce a diabetes-like effect. The level of VEGF mRNA and protein showed a significant decrease in miR200b mimic injected diabetic retinas compared to the scrambled control injected ones (FIG. 13 a), b)). On the other hand, antigomir injected non-diabetic rat retinas showed increased VEGF mRNA and protein levels (FIG. 13 a), b)).

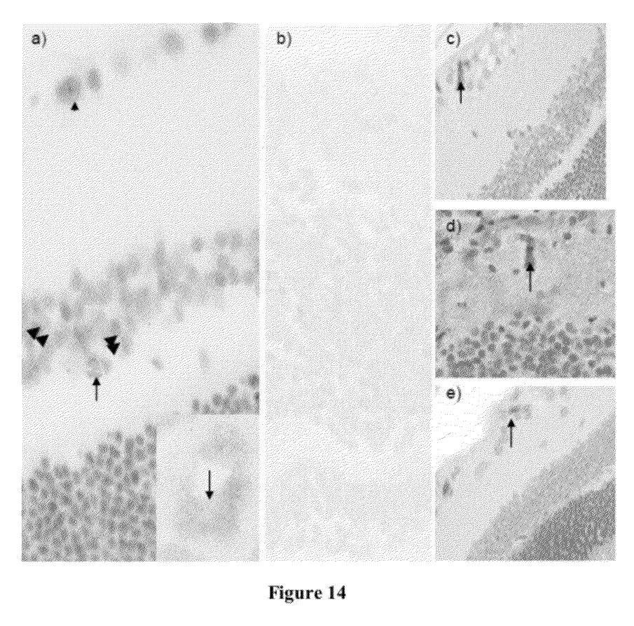

To study permeability changes, albumin permeation from the retinal vasculature was measured using an albumin immunostaining as previously described [25,32]. FIG. 14a) is a photomicrograph of a LNA™-ISH study of retinal tissues in a control rat retina showing localization of miR200b in the retinal capillaries (arrow), ganglion cells (arrowheads) and in the cells of inner nuclear layer (double arrowheads, both in the glial and neuronal elements, inset shows enlarged view of capillaries with cytoplasmic and nuclear miR200b localization (arrow)). FIG. 14 b) is a photomicrograph of a LNA™-ISH study of retinal tissues in a diabetic rat retina (in similar orientation) showing minimum (if any) expression of miR200b, indicating loss of miR200b in the retina in diabetes. FIG. 14 c) is an immunocytochemical stain on the control rat retina using anti-albumin antibody showing intra vascular albumin (arrow). FIG. 14d) similar stain as in FIG. 14 c) in the diabetic rat retina resulted in intravascular reactivity (arrow) and diffuse staining of the retina, indicating increased vascular permeability. Diabetes-induced increased vascular permeability was prevented by miR200b mimic injection. In FIG. 14 e) following intravitreal miR200b injection albumin staining was only present in the intravascular compartment (arrow). No such effects were seen following scrambled miR200b injection (not shown) (ALK Phos was used as chromogen with no counterstain in LNA-ISH; DAB chromogen and hematoxylin counterstain in albumin stain).

Glucose Induced Reduced miR200b Mediates Up-Regulation of Transcriptional Coactivator p300

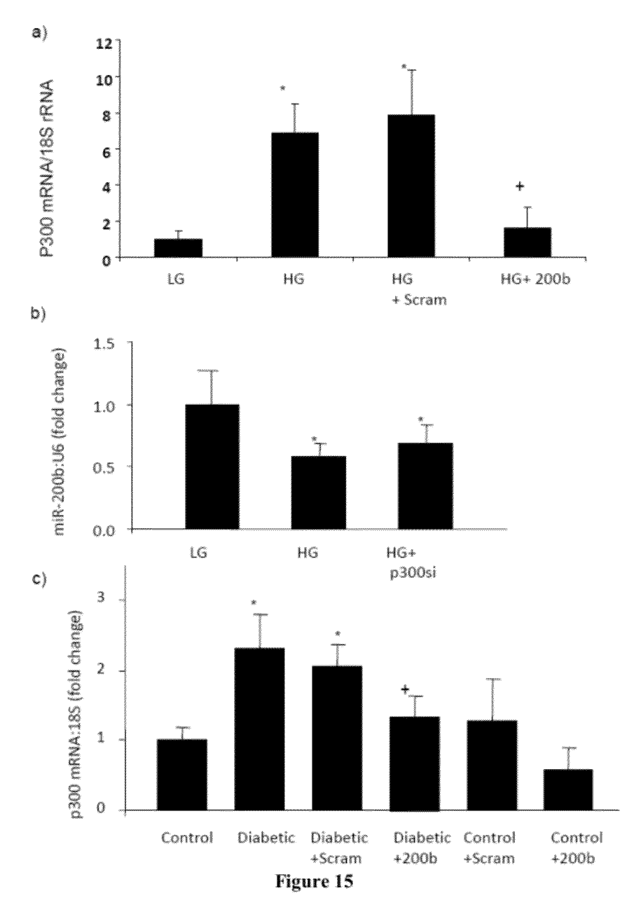

It has been shown that miR200b regulates epithelial to mesenchymal transition in malignancies by controlling p300, a transcription co-activator [35-37]. Increased p300 has been shown in DR and glucose-exposed endothelial cells (see FIG. 15 a)) [26,31,33]. The Applicants next studied whether hyperglycemia changes p300 through miR200b. The Applicants discovered that miR200b mimic transfection prevented high glucose (HG)-induced p300 up-regulation in the endothelial cells (FIG. 15 a)). However, glucose-induced down-regulation of miR200b in the endothelial cells was not corrected by p300 silencing (FIG. 15 b)). To further examine whether some of the mechanisms of miR200b's action is mediated through regulation of p300 in vivo, p300 mRNA expression in the retinal tissues was examined following intravitreal injection of miR200b mimics. As shown in FIG. 15 c) diabetes induced up-regulation of retinal p300 mRNA was prevented by miR200b injection suggesting another mechanisms by which miR200b may act on vasoactive factors.

### Example 2

Aim: To investigate whether similar alterations of miRNA along with their target alterations occur in human diabetic retinopathy. (2) To investigate whether the changes in endothelial cells or in the diabetic animals occurs in human proliferative DR. Retinal tissues from autopsy from non-diabetic and diabetic individuals with known retinopathy were collected within 6 hrs of death.

miR200b Down-Regulation is Present in Human Diabetic Retinopathy

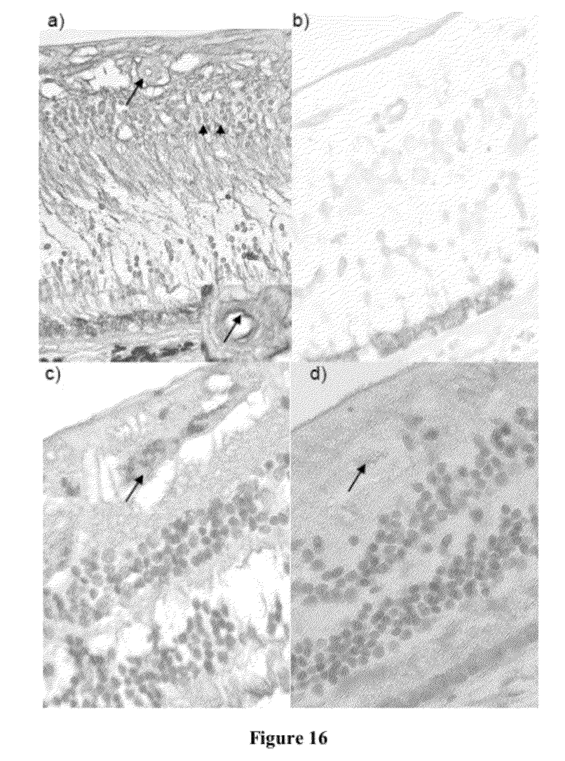

The Applicants examined human retinas in the enucleated eyes from archival sources using in situ hybridization and immunostains. Applicants found reduced miR200b and increased extravascular albumin in the retinas from diabetic human samples. The cellular distribution of miR200b was similar to the rat eyes (see micrographs of FIG. 16).

FIG. 16 a) is a photomicrograph of a LNA™-ISH study of retinal tissues from non-diabetic human retina showing localization of miR200b in the retinal capillaries (arrow), and in the cells of inner nuclear layer (double arrowheads). FIG. 16 b) is a micrograph of retinal tissues in a diabetic human retina (in similar orientation as in a)) showing minimal (if any) expression of miR200b. FIG. 16 c) is a micrograph of an immunocytochemical stain on the non diabetic human retina using anti-albumin antibody showing intra vascular albumin (arrow). FIG. 16 d) is a micrograph of diabetic human retina showed intravascular albumin staining (arrow) and diffuse staining if the retina, indicating increased vascular permeability. (ALK Phos was used as chromogen with no counterstain in LNA™-ISH; DAB chromogen and hematoxylin counterstain in albumin stain).

### Example 3

Aim: To investigate whether similar alterations of miRNA in diabetic retinopathy, along with their target alterations occur in other models of diabetes.

db/db mice (a model for type 2 diabetes mellitus) and their control mice were purchased from Jackson laboratories. Following onset of diabetes (blood glucose estimation), they were followed up for a period of two months. Metabolic parameters, body weight, urine sugar, urine ketones were monitored for two months. At the end of this period, the mice were sacrificed and retinal tissues collected. miRNA were extracted and analysed according to the methods previously provided in Example 1.

miR200b Down-Regulation is Present in Mice Diabetic Retinopathy (Model of Type 2 Diabetes)

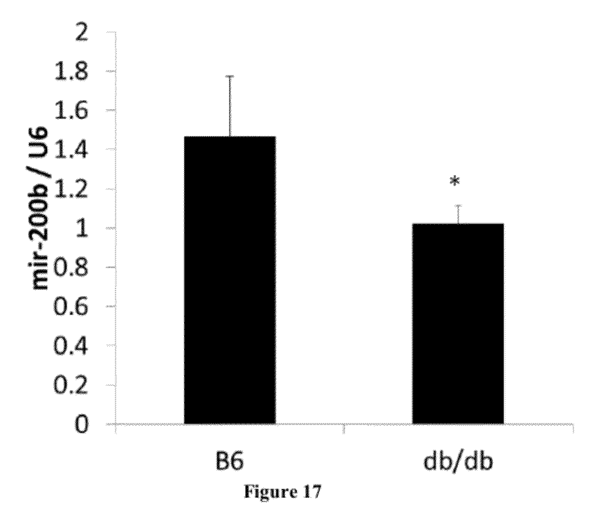

qRT-PCR analysis of the expression levels of miR200b was also studied in db/db mice retina. FIG. 17 illustrates a statistically significant decrease in the expression of miR200b in the retina of diabetic mice compared to the retinas of normal (control) mice. miR200b was found to be reduced in the retina of db/db mice (db/db) after two months of diabetes compared to age and sex matched controls (C).

miR146a Down-Regulation is Present in Mice Diabetic Retinopathy (Model of Type 2 Diabetes)

As previously shown, the Applicants further demonstrated by qRT-PCR analysis that miR146a levels in is statistically significant reduced in the retinal tissues from the db/db diabetic mice (db/db) (a model of type 2 diabetes) compared to miR146a levels in age and sex matched controls (C) (see FIG. 8).

### Example 4

### miR146a and miR320 in Human Retina

FIG. 19 illustrates amplification plots (qRT-PCR analysis) of vitreous fibrovascular tissue from two human patients with proliferative diabetic retinopathy showing presence of miR146a and miR320. The patients with proliferative diabetic retinopathy underwent vitrectomy in which fibrovascular tissue were removed from the vitreous. Using the procedures provided in Example 1, miRNA was extracted from the human retina samples and analyzed for miR146a and miR320. Very low level of miR320 and miR146a were seen. This suggests that these miRNAs are important in proliferative diabetic retinopathy and possibly reduced.

### Example 5

### miR1 in the rat Retina

miR1 is Down-Regulated in the Retina of Diabetic Animals

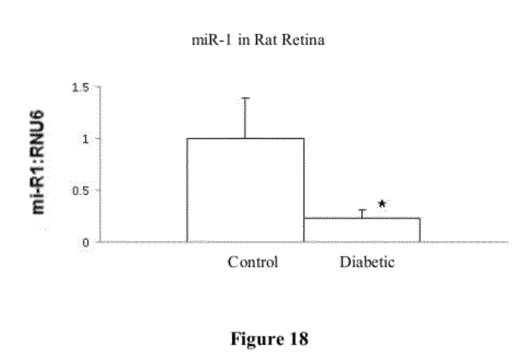

Down-regulation of miR1 in the retina of diabetic rats shown in FIG. 1 was verified with qRT-pCR. FIG. 18 illustrates a statistically significant decrease in the expression of miR1 in the retinas of diabetic rats compared to the retinas of normal (control) rats respectively. The experiments were performed similar to example 1

miR144 and miR450 Expression in Retina

Up-regulation of miR144 and miR450 in the retina of diabetic rats shown in FIG. 1 was verified with qRT-pCR. FIG. 19 a) shows a statistically significant up-regulation of miR144, and FIG. 19 b) shows a statistically significant up-regulation of miR450 in the retina of diabetic rats compared to the levels of miR144 and miR450 in retina of normal (control) rats. The experiments were performed similar to example 1.

### DISCUSSION

The Examples provided above demonstrate a novel pathway causing VEGF and FN expression and subsequent alterations in the retina in diabetes. The Applicants have shown that high levels of glucose in diabetes, causes: (a) down-regulation of miR-146a which controls fibronectin (FN) mRNA and protein levels; (b) down-regulation of miR-200b which controls VEGF mRNA and protein levels (c) down regulation of miR1 and miR320, (d) up-regulation of miR144 and miR450, and increased permeability both in vivo and in vitro. The Applicants also demonstrated that they were able to prevent diabetes-induced, FN/VEGF-mediated functional changes in the endothelial cells and in the retina by miR146a and miR-200b mimic treatment respectively.

The Applicants investigated the mechanisms at multiple levels of complexities. Following initial identification of miR1, miR146a, miR200b, miR320 down-regulation and miR144 and miR450 up-regulation in the retina in diabetes, the Applicants used HUVECs to identify the in vitro biologic significance of the miRNAs alterations. Although these HUVECs are not retinal origin, they are widely used as a model for the study of endothelial abnormalities in several diseases including DR [31,32]. However, in parallel the Applicants investigated retinal capillary endothelial cells and demonstrated similar changes as those found in HUVECs. Following in vitro studies, the Applicants also used a well established animal models of type 1 and type 2 diabetes mellitus to identify in vivo significance of the miRNA alterations. Finally the Applicants examined human retinal tissues from normal and diabetic individuals to corroborate that similar changes are also present in human retina. This constitutes the first study to investigate miRNAs in DR and directly demonstrates a functional and potential therapeutic and diagnostic implications of miR1, miR146a, miR200b, miR320, miR144 and miR450 in DR.

In keeping with the data presented herein, one previous study has previously demonstrated presence of miR-200b in human and rat retina [38]. Both this and the present study demonstrating miR200b expression in humans and rats, suggest evolutionary conservation and may reflect a possible conserved functional role within the mammalian retina. Potential role of miRNAs in non-diabetic angiogenesis has further been investigated in mice homozygous for a hypomorphic allele of Dicer. These mice lacked angiogenesis and died in utero [39]. Another study found that a nonlethal Dicer hypomorphism caused female mice to be sterile due to the failure of angiogenesis in the corpus luteum [40]. In a model of ischemic ocular neovascularisation, seven miRNAs were increased and three were decreased in the retina [41]. On the other hand, neovascularisation in DR may be different from non-diabetic neovascularisation with respect to miRNA. No alteration of miR-200b was identified in such condition [41].

Regulatory role of miR-200b with p300 is further interesting. It has been shown, that miR-200 may regulate p300, a histone aceylator and transcription co-activator in malignancies [37]. Other research indicated that in pancreatic ductal adenocarcinoma, six p300 targeting miRNAs, including miR-200b, were upregulated in the highly metastatic group. The Applicants have previously shown the role of p300 in DR and other diabetic complications and that it regulates multiple gene and protein expression in diabetes [31, 42]. Such effects of p300 are mediated by its capacity to control actions of a large number of transcription factors [26]. The present study showed a novel miR200b mediated mechanisms, by which p300 is regulated in diabetes. Hence, in addition to its direct inhibitory effects on hyperglycemia-induced VEGF expression; miRNA may mediate such effects indirectly through p300. Such p300-mediated action of miR200b may potentially affect gene expression of multiple vasoactive factors [26,31].

DR is a complex problem, in which multiple transcripts are altered, kicking off multiple abnormal pathways. Targeting individual proteins for treatments of DR have been tried for a long time and have failed in clinical trials. From a mechanistic standpoint, one miRNA regulates multiple genes, and targeting one or few miRNAs provides potential unique opportunities to prevent multiple gene expression.

The above disclosure generally describes the present invention. Changes in form and substitution of equivalents are contemplated as circumstances may suggest or render expedient. Although specific terms have been employed herein, such terms are intended in a descriptive sense and not for purposes of limitation. Other variations and modifications of the invention are possible. As such modifications or variations are believed to be within the sphere and scope of the invention as defined by the claims appended hereto.

### NON-PATENT REFERENCES

1. Chuang J C, Jones P A. Epigenetics and microRNAs. Pediatr Res 2007; 61: 24R-29R.
2. Bartel D P. MicroRNAs: genomics, biogenesis, mechanism, and function. Cell 2004; 116: 281-297.
3. Saito Y, Liang G, Egger G, Friedman J M, Chuang J C, Coetzee G A, Jones P A. Specific activation of microRNA-127 with down-regulation of the proto-oncogene BCL6 by chromatinmodifying drugs in human cancer cells. Cancer Cell 2006; 9:435-43.
4. Ouaamari A, Baroukh N, Martens G A, Lebrun P, Pipeleers D, van Obberghen E. miR375 targets 3′-phosphoinositide-dependent protein kinase-1 and regulates glucose-induced biological responses in pancreatic beta-cells. Diabetes 2008; 57(10):2708-17.
5. Wang X H, Qian R Z, Zhang W, Chen S F, Jin H M, Hu R M. MicroRNA-320 expression in myocardial microvascular endothelial cells and its relationship with insulin-like growth factor-1 in type 2 diabetic rats. Clin ExP Pharmacol Physiol. 2008; 36(2):181-188.
6. Wang Q, Wang Y, Minto A W, Wang J, Shi Q, Li X, Quigg R J. MicroRNA-377 is up-regulated and can lead to increased fibronectin production in diabetic nephropathy. FASEB J 2008; 22(12):4126-35.
7. Kato M, Zhang J, Wang M, Lanting L, Yuan H, Rossi J J, Natarajan R. MicroRNA-192 in diabetic kidney glomeruli and its function in TGF-beta-induced collagen expression via inhibition of E-box repressors. Proc Natl Acad Sci USA 2007; 104(9):3432-7.
8. Xiao J, Luo X, Lin H, Zhang Y, Lu Y, Wang N, Zhang Y, Yang B, Wang Z. MicroRNA miR133 represses HERG K+ channel expression contributing to QT prolongation in diabetic hearts. J Biol Chem 2007; 282(17):12363-7.
9. Feng B, Chen S, George B, Feng Q, Chakrabarti S. miR133a regulates cardiomyocyte hypertrophy in diabetes. Diabetes Metab Res Rev. 2010; 26:40-9.
10. van Rooij E, Sutherland L B, Liu N, Williams A H, McAnally J, Gerard R D, Richardson J A, Olson E N. A signature pattern of stress-responsive microRNAs that can evoke cardiac hypertrophy and heart failure. Proc Natl Acad Sci USA 2006; 103: 18255-60.
11. Cheng Y, Ji R, Yue I, Yang J, Liu X, Chen H, Dean D B, Zhang C. MicroRNAs are aberrantly expressed in hypertrophic heart: do they play a role in cardiac hypertrophy? Am J Pathol 2007; 170:1831-40.
12. Graham, F. L. and van der Eb, A. J., Virology 52:456-467, 1973.
13. McCutchan, J. H. and Pagano, J. S., Natl. Cancer Inst. 41:351-357, 1968.
14. Chu G, Hayakawa H, Berg P “Electroporation for the Efficient Transfection of Mammalian-Cells with DNA.” Nucleic Acids Research 15 (3): 1311-1326, 1987.
15. Fraley, R. et al., (1980), J. Biol. Chem. 255, 10431.
16. Capecchi, M. R. (1980). High efficiency transformation by direct microinjection of DNA into cultured mammalian cells. Cell 22: 479-488.
17. Felgner, P. L., Gadek, T. R. Holm, M., Roman, R., Chan, H. W., Wenz, M., Northrop, J. P., Ringold, G. M,. and Danielsen, M. Lipofection: A highly efficient, lipid-mediated DNA-transfection procedure. Proc. Natl. Acad. Sci. USA, Vol. 84, pp. 7413-7417, 1987.
18. Acheampong A A et al, 2002, Drug Metabol. and Disposition 30: 421-429.
19. Bennett J, Pakola S, Zeng Y, Maguire A M. Humoral antibody response after administration of El-deleted adenoviruses: Immune privilege of the subretinal space. Hum Gene Ther. 1996; 7:1763-1769.
Ambatia, J., and Adamis, A. P., Transscleral drug delivery to the retina and choroids. Progress in Retinal and Eye Res. 2002; 21: 145-151.
21. Cheng Y, Ji R, Yue J, et al. MicroRNAs are aberrantly expressed in hypertrophic heart: do they play a role in cardiac hypertrophy? Am J Pathol 2007; 170: 1831-1840.
22. Chen S, Evans T, Mukherjee K; Karmazyn M, Chakrabarti S. Diabetes-induced myocardial structural changes: role of endothelin-1 and its receptors. J Mol Cell Cardiol 2000; 32: 1621-1629.
23. Evans T, Deng D X, Chen S, Chakrabarti S. Endothelin receptor blockade prevents augmented extracellular matrix component mRNA expression and capillary basement membrane thickening in the retina of diabetic and galactose-fed rats. Diabetes 2000; 49: 662-666.
24. Hileeto D, Cukiernik M, Mukherjee S, et al. Contributions of endothelin-1 and sodium hydrogen exchanger-1 in the diabetic myocardium. Diabetes Metab Res Rev 2002; 18: 386-394.
25. Cukiernik M, Hileeto D, Evans T, Mukherjee S, Downey D, Chakrabarti S, Vascular endothelial growth factor in diabetes induced early retinal abnormalities. Diabetes Res Clin Pract 2004; 65:197-208.
26. Chen S, Feng B, George B, Chakrabarti R, Chen M, Chakrabarti S, Transcriptional Co-activator p300 Regulates Glucose Induced Gene Expression in the Endothelial Cells. Am J Physiol Endocrinol Metab 2010; 298: E127-E137.
27. Chen S, Mukherjee S, Chakrabarti, S. Glucose-induced endothelin dependent increased extracellular matrix protein synthesis is mediated via NF-KB and AP-1. Am J Physiol Cell Physiol 2003, 284:C263-72.
28. Xin X, Khan Z A, Chen S, Chakrabarti S. Extracellular signal-regulated kinase (ERK) in glucose-induced and endothelin-mediated fibronectin synthesis. Lab Invest 2004, 84:1451-1459.
29 Miller E C, Capps B E, Sanghani R R, Clemmons D R, Maile L A Regulation of IGF-I Signaling in Retinal Endothelial Cells by Hyperglycemia. Investigative Ophthalmology and Visual Science. 2007; 48:3878-3887.
30 van Rooij E, Sutherland L B, Qi X, Richardson J A, Hill J, Olson E N, Control of stress-dependent cardiac growth and gene expression by a microRNA. Science 2007; 316:575-579.
31 Kaur H, Chen S, Xin X, Chiu I, Khan Z A, Chakrabarti S, Diabetes-induced extracellular matrix protein expression is mediated by transcription coactivator p300. Diabetes 2006; 55:3104-3111.
32. Chen S, Apostolova M D, Cherian M G, Chakrabarti S, Interaction of endothelin-1 with vasoactive factors in mediating glucose-induced increased permeability in endothelial cells. Lab Invest 2000; 80:1311-1321.
33. Khan Z A, Chan B M, Uniyal S et al. EDB fibronectin and angiogenesis—a novel mechanistic pathway. Angiogenesis 2005; 8:183-196.
34. Wang M, Tan L P, Dijkstra M K et al. miRNA analysis in B-cell chronic lymphocytic leukaemia: proliferation centres characterized by low miR150 and high BIC/miR155 expression. J Pathol 2008; 215:13-20.
35. Adam L, Zhong M, Choi W et al. miR200 expression regulates epithelial-to-mesenchymal transition in bladder cancer cells and reverses resistance to epidermal growth factor receptor therapy. Clin Cancer Res 2009; 15:5060-5072.
36 Li Y, Vandenboom T G, Kong D et al. Up-regulation of miR200 and let-7 by natural agents leads to the reversal of epithelial-to-mesenchymal transition in gemcitabine-resistant pancreatic cancer cells. Cancer Res 2009; 69:6704-6712.
37. Mees S T, Mardin W A, Wendel C et al. EP300—a miRNA-regulated metastasis suppressor gene in ductal adenocarcinomas of the pancreas. Int J Cancer 2010; 126:114-124.
38. Arora A, McKay G J, Simpson D A. Prediction and verification of miRNA expression in human and rat retinas. Invest Ophthalmol Vis Sci 2007; 48:3962-3967.
39. Yang W J, Yang D D, Na S, Sandusky G E, Zhang Q, Zhao G. Dicer is required for embryonic angiogenesis during mouse development. J Biol Chem 2005; 280:9330-9335.
40. Otsuka M, Zheng M, Hayashi M, Lee J D, Yoshino O, Lin S, Han J. Impaired microRNA processing causes corpus luteum insufficiency and infertility in mice. J Clin Invest 2008; 118:1944-1954.
41. Shen J, Yang X, Xie B, Chen Y, Swaim M, Hackett S F, Campochiaro P A. MicroRNAs regulate ocular neovascularization. Mol Ther 2008; 16:1208-1216.
42. Chiu J, Khan Z A, Farhangkhoee H, Chakrabarti S, Curcumin prevents diabetes-associated abnormalities in the kidneys by inhibiting p300 and nuclear factor-kappaB. Nutrition 2009; 25:964-972.

1-33. (canceled)

34. A method of treating a subject of a disorder associated with glucose mediated cell damage wherein said method comprises administering to the subject at least one of (a) an agent capable of increasing the level of one or more miRNAs selected from miR-1, miR-146a, miR200b or miR-320 in a cell or cells of the subject, and (b) a miRNA inhibitory agent capable of decreasing the level of miRNA in a cell or cells of the subject, said miRNA inhibitory agent being targeted to one or more of miR-144 and miR-450 in a cell or cells of the subject.

35. The method of claim 34 wherein the agent up-regulates the expression of at least one of miR-1, miR-146a, miR200b or miR-320.

36. The method of claim 34 wherein the agent comprises a miRNA mimetic or mixture of miRNA mimetics.

37. The method of claim 34 wherein said agent is provided as a miRNA, a miRNA precursor, a mature miRNA, a DNA molecule encoding for said miRNA, miRNA precursor or mature miRNA, or any combinations thereof.

38. The method of claim 36 wherein said miRNA mimetic or mixture of miRNA mimetics is provided in a composition comprising a pharmaceutically acceptable carrier.

39. The method of claim 36 wherein said miRNA mimetic or mixture of miRNA mimetics comprises a nucleotide sequence.

40. The method of claim 34 wherein said agent is provided within a delivery vehicle.

41. The method of claim 40 wherein the delivery vehicle is selected from a viral vector, microspheres, liposomes, colloidal gold particles, lipopolysaccharides, polypeptides, polysaccharides, or pegylation of viral vehicles.

42. The method of claim 34 wherein the miRNA inhibitory agent is selected from an antagomir, an antisense RNA or a short interfering RNA, or any combinations thereof.

43. The method of claim 42 wherein said miRNA inhibitory agent is provided in a composition comprising a pharmaceutically acceptable carrier.

44. The method of claim 34 wherein the disorder is a chronic diabetic condition.

45. The method of claim 34 wherein the agent or the miRNA inhibitory agent is administered by a parenteral administration route or a topical route.

46. The method of claim 34 wherein said disorder is diabetic retinopathy, and wherein the agent or the miRNA inhibitory agent is administered by intraocular administration or topical instillation to the eye.

47. The method of claim 34 wherein said disorder is diabetic retinopathy, and wherein the agent or the miRNA inhibitory agent is administered by an ocular implant.

48. A method of treating diabetic retinopathy in a subject, wherein said method comprises administering to the subject a composition comprising one or more miRNA mimetics and a pharmaceutically active agent, said one or more miRNA mimetics comprising a nucleotide sequence selected from SEQ ID NOs: 1-4.

49. A method for diagnosing a disorder in a subject, said disorder associated with glucose mediated cell damage, wherein said method comprises measuring an expression profile of one or more miRNAs selected from miR1, miR146a, miR200b, miR320, miR144 or miR450 in a sample from the subject, wherein a difference in the miRNA expression profile of the sample from the subject and the miRNA expression profile of a normal sample or a reference sample is indicative of the disorder associated with glucose mediated cell damage.

50. The method for diagnosing a condition according to claim 49 wherein said disorder is a chronic diabetic condition, including diabetic retinopathy, diabetic nephropathy, or diabetic large vessels disease.

51. A composition for treating a disorder associated with glucose mediated cell damage comprising (a) at least one of (i) an agent capable of increasing the level of one or more of miR-1, miR-146a, miR200b or miR-320 in a cell, and (ii) a miRNA inhibitory agent capable of decreasing the level of miRNA in a cell or cells of the subject, said miRNA inhibitory agent being targeted to one or more of miR-144 and miR-450 in a cell and (b) a pharmaceutically acceptable carrier.

52. The composition of claim 51 wherein the agent up-regulates the expression of at least one of miR-1, miR-146a, miR200b or miR-320.

53. The composition of claim 51 wherein the agent comprises a miRNA mimetic or mixture of miRNA mimetics.

54. The composition of claim 51 wherein said agent is provided as a miRNA, a miRNA precursor, a mature miRNA, a DNA molecule encoding for said miRNA, miRNA precursor or mature miRNA, or any combinations thereof.

55. The composition of claim 53 wherein said miRNA mimetic or mixture of miRNA mimetics comprises a nucleotide sequence selected from SEQ ID NOs: 1-4.

56. The composition of claim 51 wherein the miRNA inhibitory agent is selected from an antagomir, an antisense RNA or a short interfering RNA, or any combinations thereof.

57. The composition of claim 51 wherein the disorder is a chronic diabetic condition.

58. The composition of claim 51 wherein the agent or the miRNA inhibitory agent is administered by a parenteral administration route or a topical route.

59. The composition of claim 51 wherein said disorder is diabetic retinopathy, and wherein the agent or the miRNA inhibitory agent is administered by intraocular administration or topical instillation to the eye.

60. The composition of claim 51 wherein said disorder is diabetic retinopathy, and wherein the agent or the miRNA inhibitory agent is administered by an ocular implant.

61. A method of treating diabetic retinopathy in a subject, wherein said method comprises administering to the subject a composition comprising a miRNA inhibitory agent targeted to one or more of miR-144 or miR-450, and a pharmaceutically active agent.
